(19) United States
(12) Patent Application Publication
(10) Pub. No.: US 2009/0317342 A1
(43) Pub. Date: Dec. 24, 2009

(54) USE OF FLAVONOIDS

(76) Inventors: Thomas Rudolph, Darmstadt (DE); Herwig Buchholz, Frankfurt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/296,680

(22) PCT Filed: Mar. 16, 2007
(86) PCT No.: PCT/EP2007/002340

§ 371 Date: May 12, 2009

(30) Foreign Application Priority Data
Apr. 13, 2006 (DE) 10 2006 017 879.3

(51) Int. Cl. A61K 8/49; A61K 47/22; A61Q 17/04; A61Q 13/00; A23L 1/00; A23L 1/48

(52) U.S. Cl. 424/59; 514/772; 514/777; 512/2; 426/654

(57) ABSTRACT

The present invention relates to the use of at least one flavonoid for odour improvement and/or odour stabilisation, and to corresponding compositions and the preparation thereof.

The present invention relates to the use of at least one flavonoid for odour improvement or odour stabilisation, and to corresponding compositions and the preparation thereof.

Everyday human life is no longer conceivable without scents, aroma substances, generally perfume oils, fragrances or aroma chemicals. Since time immemorial, they have played an important role in human culture, at first in cult uses, a little later also in beauty care. In our time too, they have an outstanding and continuously increasing importance, for example in the area of perfuming of products, in the area of body-care products, in particular also as actual “perfumes” or in the area of detergents and cleaning products.

A crucial factor for the perceptibility of a scent is its volatility, with the molecular weight also playing an important role in addition to the nature of the functional groups and the structure of the chemical compound. Thus, most scents have molecular weights up to about 200 daltons, while molecular weights of 300 daltons and above tend to represent an exception. Owing to the different volatility of scents, the odour of a perfume composed of a plurality of scents changes during evaporation, where the odour impressions are divided into “top note”, “middle note” or “body”, and “end note” or “dry out”. Since odour perception, which is very individual in humans, is also to a large part based on odour intensity, the top note of a perfume is naturally determined by highly volatile compounds, while the end note consists for the most part of less volatile, i.e. tenacious scents. In the composition of perfumes, more readily volatile scents can, for example, be bound to certain fixatives, preventing excessively rapid evaporation thereof.

In chemical terms, perfume oils are often reactive compounds of the ester, ether, aldehyde, ketone, alcohol and hydrocarbon type. In particular during storage, or in the presence of other compounds—in particular in the general field of perfuming of products—fragrances can react, or secondary odours can form due to reaction of other ingredients.

Aroma chemicals include fragrances and flavours.

The object of the present invention was accordingly to find ways of stabilising or improving the fragrance or odour of compositions or premixes.

Surprisingly, it has now been found that this is achieved by the use of at least one flavonoid.

The present invention therefore relates firstly to the use of at least one flavonoid for odour improvement and/or odour stabilisation.

The flavonoid here acts as stabiliser for perfume oils or active compounds, for example self-tanning substances, and/or reduces or prevents storage-dependent malodours of fragrances or active compounds. Furthermore, the flavonoid is able to mask unpleasant inherent odours of active compounds, in particular of cosmetic raw materials.

It is preferred in accordance with the invention for the odour improvement to take place in cosmetic, dermatological or pharmaceutical compositions, in premixes for cosmetic, dermatological or pharmaceutical compositions or in foods or food supplements.

In another, likewise preferred variant of the invention, the odour improvement or odour stabilisation takes place in perfumes.

The present invention furthermore relates to corresponding perfume compositions comprising at least one flavonoid in addition to perfume oil.

The use of flavonoids, in particular, in cosmetics or pharmacy is known per se. Thus, for example, DE-A-19739349 describes the use of troxerutin as antioxidant or free-radical scavenger in cosmetic and dermatological compositions.

For the purposes of this invention, the term “flavonoid” encompasses, in particular, compounds which, owing to their basic structure, can be assigned to the following groups:
chalcones
chromones
aurones
flavanones
flavan-3-ols (catechols)
flavones
isoflavones
flavan-3,4-diols (leukoanthocyanidines)
flavonols (3-hydroxyflavan-4-one)
flavanonols

The name “flavonoid” is derived from the Latin word flavus=yellow and thus takes into account the fact that most of these substances have a yellowish colour in their pure form.

The following flavonoids should be mentioned by way of example: 5-hydroxy-7,4′-dimethoxyflavone 8-sulfate, 7,8-dihydroxyflavone, luteolin (flavones); catechol, epicatechol, epigallocatechol gallate (EGCG, TEAVIGO® DSM) (flavan-3-ols or flavan-3-ol derivatives); kaempferol (flavonol); taxifolin (flavanonol), the iso-flavones from the group isoflavone, daidzein, genistein, prunetin, biochanin A, orobol, santal or pratensein, as well as naringenin (flavanone), and glycosides of naringenin, for example naringin 7-neohesperidoside.

Preferred flavonoids are derived from the following groups:
flavonols
flavonol o-glycosides
flavonol o-glycoside-containing extracts

Flavonoids usually occur as soluble glycosides in the cell sap of plants. The preferred flavonoids also include aglycones (sugar-free structures) and aglycone conjugates. Possible aglycone conjugates are hydroxyl derivatives, where all or some of the hydroxyl groups are alkylated, methylated, glycylated, sulfated or esterified. Besides hydroxyl derivatives, C derivatives are also suitable as aglycone conjugates.

For the group of the flavonols, particular preference is given to the aglycone quercetin.

In the group of the flavonol o-glycosides, the flavonol 3-glycosides, such as rutin, α-glucosylrutin, tiliroside, isoquercetin, rutin sulfate, trishydroxyethylrutin (troxerutin), and sulfates and phosphates thereof are particularly preferred. Flavonol 7- and 8-glycosides can also be used.

The term “rutin sulfate” encompasses mono-, di-, tri-, tetra- or polysulfates of rutin and mixtures of these rutin sulfates. The term “troxerutin” encompasses mono-, di-, tri-, tetra- or polyethoxylates of rutin and mixtures of these rutin ethoxylates.

In accordance with the invention, preference is given to the use of the flavonoids selected from the group quercetin, rutin, rutin sulfate, α-glucosylrutin, tiliroside, troxerutin and/or isoquercetin.

For the group of the flavonol or flavonol o-glycoside-containing extracts, the active compound combinations emblica, liquorice and/or horse chestnut extract are preferred. Emblica is obtained from the fruit of the deciduous tree Phyllanthus emblica (also Emblica officinalis), for example in India, China, Pakistan or Nepal. The principal ingredients of emblica are the low-molecular-weight tannic acids emblicanin A and B, which bind the iron occurring in the skin in the form of complexes. Preferred emblica solutions are commercially available, for example as EMBLICA® (MERCK) or CAPROS® (see, for example, U.S. Pat. No. 6,235,721 or U.S. Pat. No. 6,124,268). In principle, all emblica mixtures, alone or in combination with at least one flavonoid, are suitable for a use according to the invention.

The liquorice extract contains the flavonoid glabridin (a stearyl glycyrhetinate) and/or licochalcone A.

The horse chestnut extract contains, for example, esculin and other flavonol and/or flavonol glycoside constituents.

Plant substance mixtures of this type can be obtained in a manner familiar to the person skilled in the art, for example by pressing or extraction of the corresponding plants.

For the use according to the invention in corresponding formulations, the at least one flavonoid is preferably employed in a total amount of 0.01 to 10% by weight, more preferably in an amount of 0.1 to 5% by weight. For the use according to the invention in premixes for corresponding formulations, the at least one flavonoid is preferably employed in a total amount of 1 to 95% by weight, preferably in amounts of 10 to 50% by weight or 1 to 10% by weight, particularly preferably in amounts of 15 to 33% by weight or 1.5 to 5% by weight. Which preferred parameter range is employed in the premix depends on the use of the premix or the use of the formulation comprising the premix. The corresponding choice can be made by the corresponding person skilled in the art with the aid of his expert knowledge.

For the purposes of the present invention, the terms preparation and formulation are also used synonymously in addition to the term composition.

For the purposes of the present invention, the term fragrance is also used synonymously in addition to the term perfume oil.

A premix for a composition or formulation is taken to mean a blend of solids of, for example, pulverulent ingredients, which is mixed, as a powder mixture as such, with at least one suitable vehicle and optionally further ingredients to give a composition.

However, the at least one flavonoid can also be employed in accordance with the invention in premixes whose physical state at room temperature is not solid or pulverulent, for example comprising a liquid component, whose premix with other components may itself, however, again be either solid or liquid.

A composition is taken to mean a mixture of the at least one flavonoid used in accordance with the invention together with the component whose odour is to be improved and/or stabilised and at least one vehicle which is known for cosmetic, dermatological or pharmaceutical purposes.

It may be preferred here in accordance with the invention for the at least one flavonoid to be a flavonoid in which one or more phenolic hydroxyl groups have been blocked by etherification or esterification. For example, hydroxyethyl-substituted flavonoids, such as, preferably, troxerutin, troxequercetin, troxeisoquercetin or troxeluteolin, and flavonoid sulfates or flavonoid phosphates, such as, preferably, rutin sulfates, have proven to be particularly suitable flavonoids for the purposes of the present invention.

For the purposes of the use according to the invention, particular preference is given to rutin sulfate and troxerutin. Very particular preference is given to the use of troxerutin.

Flavonoids which are preferred in accordance with the invention furthermore have a non-positively charged flavan skeleton. It is thought that these flavonoids complex metal ions, such as, for example, Fe²⁺/Cu²⁺, and thus prevent or reduce auto oxidation processes in fragrances or compounds whose degradation results in malodours.

Further preferred combinations of embodiments are disclosed in the claims.

For the purposes of this invention, the term perfume oil is very broadly drawn since it encompasses here all individual substances or substance mixtures which trigger an odour perception, preferably a pleasant one, in humans and are therefore suitable and widely used for perfuming, for example, articles such as industrial and sanitary articles, soaps, cosmetics (body-care compositions), pharmaceuticals, foods or food supplements and the like. For the purposes of this invention, the term perfume oil consequently essentially encompasses all essential oils, scents and aroma substances, individually and in particular in mixtures of two or more different ingredients. However, essences and aromas or aroma substances or mixtures thereof also expressly fall under the term perfume oils here. In addition, the term perfume oil(s) also explicitly encompasses pheromones, although these are not scents in the actual sense. In particular, the term perfume oils is of course taken to mean the classical perfume oils, i.e. those which are obtained, for example, by pressing fruit skins, extraction of resinoids from resins, balsams, lichens and mosses, destraction of (petal) fragrances by means of supercritical gases or by steam distillation of previously prepared comminuted plant parts, such as, for example, rose oil.

Perfume oils which can be used are, for example, individual scent compounds, for example the synthetic products of the ester, ether, aldehyde, ketone, alcohol and hydrocarbon type. Scent compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenyl glycinate, allylcyclohexyl propionate, styrallyl propionate and benzyl salicylate. The ethers include, for example, benzyl ethyl ether, the aldehydes include, for example, the linear alkanals having 8-18 C atoms, vanillin, citral a, citral b, citronellal, citronellyloxyacetaldehyde, cyclamenaldehyde, hydroxycitronellal, lilial and bourgeonal, the ketones include, for example, the jonones, α-isomethylionone and methyl cedryl ketone, the alcohols include anethol, citronellol, eugenol, geraniol, linalool, phenylethyl alcohol and terpineol, the hydrocarbons include principally the terpenes and balsams. However, preference is given to the use of mixtures of various scents which together produce an attractive fragrance.

The perfume oils can of course also comprise natural scent mixtures, as are accessible from vegetable or animal sources, for example pine, citrus, jasmine, lily, patchouli, rose or ylang-ylang oil. Essential oils of lower volatility, which are mostly used as aroma components, are also suitable as perfume oils, for example sage oil, camomile oil, clove oil, melissa oil, mint oil, cinnamon leaf oil, lime blossom oil, juniper berry oil, vetiver oil, olibanum oil, galbanum oil and ladanum oil.

In the following division of scents into “relatively volatile” and “tenacious” scents, nothing is thus stated regarding the odour impression and regarding whether the corresponding scent is perceived as a top note or body note. Tenacious scents which can be employed for the purposes of the present invention are, for example, the essential oils, such as angelica root oil, aniseed oil, arnica blossom oil, basil oil, bay oil, bergamot oil, champaca flower oil, silver fir oil, silver fir cone oil, elemi oil, eucalyptus oil, fennel oil, spruce needle oil, galbanum oil, geranium oil, gingergrass oil, guaiacum wood oil, gurjun balsam oil, helichrysum oil, Ho oil, ginger oil, iris oil, cajuput oil, calamus oil, camomile oil, camphor oil, cananga oil, cardamom oil, cassia oil, pine needle oil, copaiba balsam oil, coriander oil, spearmint oil, caraway oil, cumin oil, lavender oil, lemon grass oil, lime oil, mandarin oil, melissa oil, musk seed oil, myrrh oil, clove oil, neroli oil, niaouli oil, olibanum oil, orange oil, oregano oil, palmarosa oil, patchouli oil, Peru balsam oil, petitgrain oil, pepper oil, peppermint oil, pimento oil, pine oil, rose oil, rosemary oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, lemon oil and cypress oil.

However, the higher-boiling or solid scents of natural or synthetic origin can also be employed for the purposes of the present invention as tenacious scents or scent mixtures, i.e. fragrances. These compounds include the compounds mentioned below and mixtures thereof: ambrettolide, α-amylcinnamaldehyde, anethol, anisaldehyde, anis alcohol, anisole, methyl anthranilate, acetophenone, benzyl acetone, benzaldehyde, ethyl benzoate, benzophenone, benzyl alcohol, benzyl acetate, benzyl benzoate, benzyl formate, benzyl valerate, borneol, bornyl acetate, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, methyl heptincarboxylate, heptaldehyde, hydroquinone dimethyl ether, hydroxycinnamaldehyd, hydroxycinnamic alcohol, indole, iron, isoeugenol, isoeugenol methyl ether, isosafrol, jasmone, camphor, carvacrol, carvone, p-cresol methyl ether, coumarin, p-methoxyacetophenone, methyl n-amyl ketone, methyl methylanthranilate, p-methylacetophenone, methylchavicol, p-methylquinoline, methyl β-naphthyl ketone, methyl-n-nonylacetaldehyde, methyl n-nonyl ketone, muscone, β-naphthol ethyl ether, β-naphthol methyl ether, nerol, nitrobenzene, n-nonyl aldehyde, nonyl alcohol, n-octyl aldehyde, p-oxyacetophenone, pentadecanolide, β-phenylethyl alcohol, phenylacetaldehyde dimethyl acetal, phenylacetic acid, pulegone, safrol, isoamyl salicylate, methyl salicylate, hexyl salicylate, cyclohexyl salicylate, santalol, scatol, terpineol, thymene, thymol, γ-undelactone, vanillin, veratrumaldehyde, cinnamaldehyde, cinnamic alcohol, cinnamic acid, ethyl cinnamate, benzyl cinnamate.

The relatively volatile scents include, in particular, the relatively low-boiling scents of natural or synthetic origin, which can be employed alone or in mixtures. Examples of relatively volatile scents are alkyl isothiocyanates (alkylmustard oils), butanedione, limonene, linalool, linayl acetate and propionate, menthol, menthone, methyl-n-heptenone, phellandrene, phenylacetaldehyde, terpinyl acetate, citral, citronellal.

It is particularly preferred in accordance with the invention for the composition to comprise at least one scent or active compound of the aldehyde or ketone type, since it has been observed that flavonoids are able to stabilise such compounds in a particular manner. The odour improvement of a composition comprising dihydroxyacetone or vanillin is particularly significant.

The odour improvement takes place in accordance with the invention in compositions or premixes for compositions which comprise at least one flavonoid having an uncharged flavan skeleton and at least one active substance which is stabilised by the presence of the flavonoid. The corresponding degradable active substances or degradation products thereof often result in malodours in compositions. Preferred active substances here again have aldehyde or ketone functions. The active substance or active compound employed in a variant of the invention is preferably at least one self-tanning substance.

Self-tanning substances which can be employed are, inter alia:

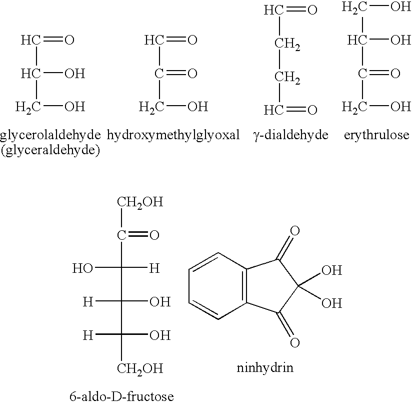

Mention should furthermore be made of 5-hydroxy-1,4-naphthoquinone (juglone), which is extracted from the shells of fresh walnuts,

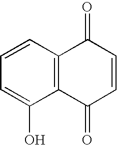

5-hydroxy-1,4-naphthoquinone (juglone) and 2-hydroxy-1,4-naphthoquinone (lawsone), which occurs in henna leaves,

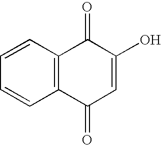

2-hydroxy-1,4-naphthoquinone (lawsone).

Preference is given to the use of the following trioses and tetroses:

1,3-dihydroxyacetone (DHA or also dihydroxyacetone) and derivatives thereof, glyceraldehyde, dihydroxyacetone phosphate, glyceraldehyde phosphate, erythrose and 1,3,4-trihydroxy-2-butanone (erythrulose). Very particular preference is given to erythrulose and/or 1,3-dihydroxyacetone (DHA), stabilised in premixes or compositions. DHA is a trifunctional ketosugar which occurs in the human body.

It may also be particularly preferred for the purposes of the present invention to employ premixes for compositions comprising, in accordance with the invention, at least one flavonoid and one or more of the self-tanning substances described here. In such premixes, the self-tanning substance to flavonoid ratio is preferably 5:1 to 1:2. Corresponding premixes are described in the German patent application with the application file reference DE 102005035683.4, and US patent applications with the file references 60/702,983 and 60/748,588, the contents of which in this respect expressly also belong to the disclosure content of the present application.

A further corresponding premix is, for example, also a solid blend comprising at least one self-tanning substance, for example DHA and/or erythrulose, a formaldehyde scavenger and, in accordance with the invention, the flavonoid. Suitable formaldehyde scavengers are, for example, D-glucitol, glycerol, 1,2-propanediol, isopropyl alcohol, ethylene glycol, diethylene glycol, resorcinol, pyrogallol, phloroglucin, hydrogensulfites, sulfites, mixtures of sulfites and sulfides, bisulfites, transresveratrol, urea, benzotriazoles or wheat gluten, in particular bisulfites. A known solid blend in this sense is DHA Plus, a self-tanning premix marketed by Merck, Darmstadt, which consists of dihydroxyacetone, sodium bisulfite and magnesium stearate, to which a flavonoid can be added in accordance with the invention for odour improvement.

In accordance with the invention, a flavonoid can also be added to premixes or compositions which comprise at least one self-tanning substance and dimethylisosorbide.

In the premixes described, which additionally comprise, in accordance with the invention, at least one flavonoid, pigments may furthermore also be present, where the layer structure of the pigments is not limited.

Advantageous coloured pigments are, for example, titanium dioxide, mica, iron oxides (for example Fe₂O₃, Fe₃O₄, FeO(OH)) and/or tin oxide. Advantageous dyes are, for example, carmine, Berlin Blue, Chromium Oxide Green, Ultramarine Blue and/or Manganese Violet. It is particularly advantageous to select the dyes and/or coloured pigments from the following list. The Colour Index numbers (CINs) are taken from the Rowe Colour Index, 3rd Edition, Society of Dyers and Colourists, Bradford, England, 1971.

| Chemical or other name | CIN | Colour |
|---|---|---|
| Pigment Green | 10006 | Green |
| Acid Green 1 | 10020 | Green |
| 2,4-Dinitrohydroxynaphthalene-7-sulfonic acid | 10316 | Yellow |
| Pigment Yellow 1 | 11680 | Yellow |
| Pigment Yellow 3 | 11710 | Yellow |
| Pigment Orange 1 | 11725 | Orange |
| 2,4-Dihydroxyazobenzene | 11920 | Orange |
| Solvent Red 3 | 12010 | Red |
| 1-(2′-Chloro-4′-nitro-1′-phenylazo)-2- | 12085 | Red |
| hydroxynaphthalene | | |
| Pigment Red 3 | 12120 | Red |
| Ceres Red; Sudan Red; Fat Red G | 12150 | Red |
| Pigment Red 112 | 12370 | Red |
| Pigment Red 7 | 12420 | Red |
| Pigment Brown 1 | 12480 | Brown |
| 4-(2′-Methoxy-5′-sulfonyldiethylamide-1′-phenylazo)- | 12490 | Red |
| 3-hydroxy-5″-chloro-2″,4″-dimethoxy-2-naphthanilide | | |
| Disperse Yellow 16 | 12700 | Yellow |
| 1-(4-Sulfo-1-phenylazo)-4-aminobenzene-5-sulfonic | 13015 | Yellow |
| acid | | |
| 2,4-Dihydroxyazobenzene-4′-sulfonic acid | 14270 | Orange |
| 2-(2,4-Dimethylphenylazo-5-sulfonyl)-1-hydroxy- | 14700 | Red |
| naphthalene-4-sulfonic acid | | |
| 2-(4-Sulfo-1-naphthylazo)-1-naphthol-4-sulfonic acid | 14720 | Red |
| 2-(6-Sulfo-2,4-xylylazo)-1-naphthol-5-sulfonic acid | 14815 | Red |
| 1-(4′-Sulfophenylazo)-2- | 15510 | Orange |
| hydroxynaphthalene | | |
| 1-(2-Sulfonyl-4-chloro-5-carboxy-1-phenylazo)-2- | 15525 | Red |
| hydroxynaphthalene | | |
| 1-(3-Methylphenylazo-4-sulfonyl)-2- | 15580 | Red |
| hydroxynaphthalene | | |
| 1-(4′,(8′)-Sulfonylnaphthylazo)-2-hydroxynaphthalene | 15620 | Red |
| 2-Hydroxy-1,2′-azonaphthalene-1′-sulfonic acid | 15630 | Red |
| 3-Hydroxy-4-phenylazo-2-naphthylcarboxylic acid | 15800 | Red |
| 1-(2-Sulfo-4-methyl-1-phenylazo)-2- | 15850 | Red |
| naphthylcarboxylic acid | | |
| 1-(2-Sulfo-4-methyl-5-chloro-1-phenylazo)-2-hydroxy- | 15865 | Red |
| naphthalene-3-carboxylic acid | | |
| 1-(2-Sulfo-1-naphthylazo)-2-hydroxynaphthalene-3- | 15880 | Red |
| carboxylic acid | | |
| 1-(3-Sulfo-1-phenylazo)-2-naphthol-6-sulfonic acid | 15980 | Orange |
| 1-(4-Sulfo-1-phenylazo)-2-naphthol-6-sulfonic acid | 15985 | Yellow |
| Allura Red | 16035 | Red |
| 1-(4-Sulfo-1-naphthylazo)-2-naphthol-3,6-disulfonic | 16185 | Red |
| acid | | |
| Acid Orange 10 | 16230 | Orange |
| 1-(4-Sulfo-1-naphthylazo)-2-naphthol-6,8-disulfonic | 16255 | Red |
| acid | | |
| 1-(4-Sulfo-1-naphthylazo)-2-naphthol-3,6,8-trisulfonic | 16290 | Red |
| acid | | |
| 8-Amino-2-phenylazo-1-naphthol-3,6-disulfonic acid | 17200 | Red |
| Acid Red 1 | 18050 | Red |
| Acid Red 155 | 18130 | Red |
| Acid Yellow 121 | 18690 | Yellow |
| Acid Red 180 | 18736 | Red |
| Acid Yellow 11 | 18820 | Yellow |
| Acid Yellow 17 | 18965 | Yellow |
| 4-(4-Sulfo-1-phenylazo)-1-(4-sulfophenyl)-5-hydroxy- | 19140 | Yellow |
| pyrazolone-3-carboxylic acid | | |
| Pigment Yellow 16 | 20040 | Yellow |
| 2,6-(4′-Sulfo-2″,4″-dimethyl)bisphenylazo)-1,3- | 20170 | Orange |
| dihydroxy-benzene | | |
| Acid Black 1 | 20470 | Black |
| Pigment Yellow 13 | 21100 | Yellow |
| Pigment Yellow 83 | 21108 | Yellow |
| Solvent Yellow | 21230 | Yellow |
| Acid Red 163 | 24790 | Red |
| Acid Red 73 | 27290 | Red |
| 2-[4′-(4″-Sulfo-1″-phenylazo)-7′-sulfo-1′- | 27755 | Black |
| naphthylazo]-1-hydroxy-7-aminonaphthalene-3,6- | | |
| disulfonic acid | | |
| 4-[4″-Sulfo-1″-phenylazo)-7′-sulfo-1′-naphthylazo]-1- | 28440 | Black |
| hydroxy-8-acetylaminonaphthalene-3,5-disulfonic acid | | |
| Direct Orange 34, 39, 44, 46, 60 | 40215 | Orange |
| Food Yellow | 40800 | Orange |
| trans-β-Apo-8′-carotene aldehyde (C₃₀) | 40820 | Orange |
| trans-Apo-8′-carotinic acid (C₃₀) ethyl ester | 40850 | Orange |
| Canthaxanthine | 40850 | Orange |
| Acid Blue 1 | 42045 | Blue |
| 2,4-Disulfo-5-hydroxy-4′-4″- | 42051 | Blue |
| bis(diethylamino)triphenylcarbinol | | |
| 4-[(-4-N-Ethyl-p-sulfobenzylamino)phenyl- | 42053 | Green |
| (4-hydroxy-2-sulfophenyl)(methylene)-1- | | |
| (N-ethyl-N-p-sulfobenzyl)-2,5-cyclohexadienimine] | | |
| Acid Blue 7 | 42080 | Blue |
| (N-Ethyl-p-sulfobenzylamino)phenyl-(2-sulfophenyl)- | 42090 | Blue |
| methylene-(N-ethyl-N-p-sulfobenzyl)-Δ^{2,5}- | | |
| cyclohexadienimine | | |
| Acid Green 9 | 42100 | Green |
| Diethyldisulfobenzyldi-4-amino-2-chlorodi-2-methyl- | 42170 | Green |
| fuchsonimmonium | | |
| Basic Violet 14 | 42510 | Violet |
| Basic Violet 2 | 42520 | Violet |
| 2′-Methyl-4′-(N-ethyl-N-m-sulfobenzyl)amino-4″-(N- | 42735 | Blue |
| diethyl)-amino-2-methyl-N-ethyl-N-m-sulfobenzyl- | | |
| fuchsonimmonium | | |
| 4′-(N-Dimethyl)amino-4″-(N-phenyl)aminonaphtho-N- | 44045 | Blue |
| dimethylfuchsonimmonium | | |
| 2-Hydroxy-3,6-disulfo-4,4′-bisdimethylaminonaphtho- | 44090 | Green |
| fuchsonimmonium | | |
| Acid Red 52 | 45100 | Red |
| 3-(2′-Methylphenylamino)-6-(2′-methyl-4′-sulfophenyl- | 45190 | Violet |
| amino)-9-(2″-carboxyphenyl)xanthenium salt | | |
| Acid Red 50 | 45220 | Red |
| Phenyl-2-oxyfluorone-2-carboxylic acid | 45350 | Yellow |
| 4,5-Dibromofluorescein | 45370 | Orange |
| 2,4,5,7-Tetrabromofluorescein | 45380 | Red |
| Solvent Dye | 45396 | Orange |
| Acid Red 98 | 45405 | Red |
| 3′,4′,5′,6′-Tetrachloro-2,4,5,7-tetrabromofluorescein | 45410 | Red |
| 4,5-Diiodofluorescein | 45425 | Red |
| 2,4,5,7-Tetraiodofluorescein | 45430 | Red |
| Quinophthalone | 47000 | Yellow |
| Quinophthalonedisulfonic acid | 47005 | Yellow |
| Acid Violet 50 | 50325 | Violet |
| Acid Black 2 | 50420 | Black |
| Pigment Violet 23 | 51319 | Violet |
| 1,2-Dioxyanthraquinone, calcium/aluminium complex | 58000 | Red |
| 3-Oxypyrene-5,8,10-sulfonic acid | 59040 | Green |
| 1-Hydroxy-4-N-phenylaminoanthraquinone | 60724 | Violet |
| 1-Hydroxy-4-(4′-methylphenylamino)anthraquinone | 60725 | Violet |
| Acid Violet 23 | 60730 | Violet |
| 1,4-Di(4′-methylphenylamino)anthraquinone | 61565 | Green |
| 1,4-Bis(o-sulfo-p-toluidino)anthraquinone | 61570 | Green |
| Acid Blue 80 | 61585 | Blue |
| Acid Blue 62 | 62045 | Blue |
| N,N′-Dihydro-1,2,1′,2′-anthraquinonazine | 69800 | Blue |
| Vat Blue 6; Pigment Blue 64 | 69825 | Blue |
| Vat Orange 7 | 71105 | Orange |
| Indigo | 73000 | Blue |
| Indigodisulfonic acid | 73015 | Blue |
| 4,4′-Dimethyl-6,6′-dichlorothioindigo | 73360 | Red |
| 5,5′-Dichloro-7,7′-dimethylthioindigo | 73385 | Violet |
| Quinacridone Violet 19 | 73900 | Violet |
| Pigment Red 122 | 73915 | Red |
| Pigment Blue 16 | 74100 | Blue |
| Phthalocyanines | 74160 | Blue |
| Direct Blue 86 | 74180 | Blue |
| Chlorinated phthalocyanines | 74260 | Green |
| Natural Yellow 6, 19; Natural Red 1 | 75100 | Yellow |
| Bixin, Nor-Bixin | 75120 | Orange |
| Lycopene | 75125 | Yellow |
| trans-alpha-, -beta- or -gamma-carotene | 75130 | Orange |
| Keto and/or hydroxyl derivatives of carotene | 75135 | Yellow |
| Guanine or pearlescent agent | 75170 | White |
| 1,7-Bis(4-hydroxy-3-methoxyphenyl)-1,6- | 75300 | Yellow |
| heptadiene-3,5-dione | | |
| Complex salt (Na, Al, Ca) of carminic acid | 75470 | Red |
| Chlorophyll a and b; copper compounds of | 75810 | Green |
| chlorophylls and chlorophyllines | | |
| Aluminium | 77000 | White |
| Aluminium hydroxide | 77002 | White |
| Water-containing aluminium silicates | 77004 | White |
| Ultramarine | 77007 | Blue |
| Pigment Red 101 and 102 | 77015 | Red |
| Barium sulfate | 77120 | White |
| Bismuth oxychloride and mixtures thereof with mica | 77163 | White |
| Calcium carbonate | 77220 | White |
| Calcium sulfate | 77231 | White |
| Carbon | 77266 | Black |
| Pigment Black 9 | 77267 | Black |
| Carbo medicinalis vegetabilis | 77268:1 | Black |
| Chromium oxide | 77288 | Green |
| Chromium oxide, water-containing | 77278 | Green |
| Pigment Blue 28, Pigment Green 14 | 77346 | Green |
| Pigment Metal 2 | 77400 | Brown |
| Gold | 77480 | Brown |
| Iron oxides and hydroxides | 77489 | Orange |
| Iron oxide | 77491 | Red |
| Iron oxide hydrate | 77492 | Yellow |
| Iron oxide | 77499 | Black |
| Mixtures of iron(II) and iron(III) hexacyanoferrate | 77510 | Blue |
| Pigment White 18 | 77713 | White |
| Manganese ammonium diphosphate | 77742 | Violet |
| Manganese phosphate; Mn₃(PO₄)₂•7H₂O | 77745 | Red |
| Silver | 77820 | White |
| Titanium dioxide and mixtures thereof with mica | 77891 | White |
| Zinc oxide | 77947 | White |
| 6,7-Dimethyl-9-(1′-D-ribityl)isoalloxazine, lactoflavin | | Yellow |
| Sugar dye | | Brown |
| Capsanthin, capsorubin | | Orange |
| Betanin | | Red |
| Benzopyrylium salts, anthocyans | | Red |
| Aluminium, zinc, magnesium and calcium stearate | | White |
| Bromothymol Blue | | Blue |

Particular preference is given to the types of pearlescent pigment listed below:
1. Natural pearlescent pigments, such as, for example,
   “pearl essence” (guanine/hypoxanthine mixed crystals from fish scales) and
   “mother-of-pearl” (ground mussel shells)
2. Monocrystalline pearlescent pigments, such as, for example, bismuth oxychloride (BiOCI)
3. Layered substrate pigments: for example mica/metal oxide

The basis for pearlescent pigments is formed by, for example, pulverulent pigments or castor oil dispersions of bismuth oxychloride and/or titanium dioxide as well as bismuth oxychloride and/or titanium dioxide on mica. The lustre pigment listed under CIN 77163, for example, is particularly advantageous.

Also advantageous are, for example, the following pearlescent pigment types based on mica/metal oxide:

| | Coating/layer | |
|---|---|---|
| Group | thickness | Colour |
| Silver-white | TiO₂: 40-60 nm | silver |
| pearlescent pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Coloured lustre pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Berlin Blue | dark blue |

Particular preference is given to, for example, the pearlescent pigments available from Merck under the trade names Timiron®, Colorona®, Dichrona®, Xirona® or Ronastar®.

The list of the said pearlescent pigments is of course not intended to be limiting. Pearlescent pigments which are advantageous for the purposes of the present invention can be obtained by numerous routes known per se. For example, other substrates apart from mica can also be coated with further metal oxides, such as, for example, silica and the like. For example, TiO₂— and Fe₂O₃-coated SiO₂ particles (“Ronasphere” grades), which are marketed by Merck and are particularly suitable for the optical reduction of fine wrinkles, are advantageous.

It may additionally be advantageous to completely omit a substrate such as mica. Particular preference is given to pearlescent pigments prepared using SiO₂. Such pigments, which may additionally also have goniochromatic effects, are available, for example, from BASF under the trade name Sicopearl Fantastico.

It may also be advantageous to employ Engelhard/Mearl pigments based on calcium sodium borosilicate coated with titanium dioxide. These are available under the name Reflecks®. Due to their particle size of 40-80 μm, they have a glitter effect in addition to the colour.

Also particularly advantageous are effect pigments available from Flora Tech under the trade name Metasomes® Standard/Glitter in various colours (yellow, red, green, blue). The glitter particles here are in the form of mixtures with various assistants and dyes (such as, for example, the dyes with the colour index (CI) numbers 19140, 77007, 77289, 77491).

Particularly suitable pigments in the premixes are, for example, Ronastar® Silver or Colorona® Bronze.

When used on the human skin, self-tanner compositions, in particular those which comprise dihydroxyacetone, tend towards malodours—presumably caused by products of side reactions—which are in some cases perceived by users as unpleasant. It has been found that these malodours are avoided on use of the flavonoids to be employed in accordance with the invention. The present invention therefore furthermore relates to the uses of at least one flavonoid for the stabilisation of and/or improvement in the odour of self-tanner compositions comprising at least one self-tanner substance on the skin.

The additional active substances preferably to be employed furthermore include, for example, UV filters, chromone derivatives, aryl oximes and parabens.

Parabens are 4-hydroxybenzoic acid esters which are used in free form or as sodium salts for the preservation of compositions in the area of foods, cosmetics and medicaments. The action of the esters is directly proportional to the chain length of the alkyl radical, but on the other hand the solubility decreases with increasing chain length. As non-dissociating compounds, the esters are predominantly pH-independent and act in a pH range from 3.0-8.0. The antimicrobial action mechanism is based on damage of the microbe membranes by the surface activity of the PHB esters and on protein denaturing. In addition, interactions occur with coenzymes. The action is directed against fungi, yeasts and bacteria. The most important parabens as preservatives are methyl 4-hydroxybenzoate, ethyl 4-hydroxybenzoate, propyl 4-hydroxybenzoate and butyl 4-hydroxybenzoate.

Of the aryl oximes, preference is given to the use of 2-hydroxy-5-methyllaurophenone oxime, which is also known as HMLO, LPO or F5. Its suitability for use in cosmetic compositions is disclosed, for example, in DE 41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and other allergic and/or inflammatory diseases of the skin and skin appendages. Compositions according to the invention which, in addition to the said compound(s), additionally comprise an aryl oxime, preferably 2-hydroxy-5-methyllaurophenone oxime, exhibit surprising anti-inflammatory suitability. The compositions here preferably comprise 0.01 to 10% by weight of the aryl oxime, it being particularly preferred for the composition to comprise 0.05 to 5% by weight of aryl oxime.

For the purposes of the invention, flavone derivatives are taken to mean flavonoids and coumaranones. Of the coumaranones, 4,6,3′,4′-tetrahydroxybenzylcoumaranone-3 is preferred.

Chromone derivatives are preferably taken to mean certain chromen-2-one derivatives which are suitable as active compounds for the preventative treatment of human skin and human hair against ageing processes and damaging environmental influences. They simultaneously exhibit a low irritation potential for the skin, have a positive effect on water binding in the skin, maintain or increase the elasticity of the skin and thus promote smoothing of the skin. These compounds preferably conform to the following formula

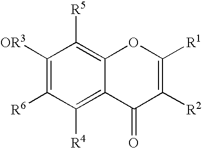

where

R¹ and R² may be identical or different and are selected from
H, —C(═O)—R⁷, —C(═O)—OR⁷,
straight-chain or branched C₁- to C₂₀-alkyl groups,
straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ— groups, where n=1 to 3,

R³ stands for H or straight-chain or branched C₁- to C₂₀-alkyl groups,

R⁴ stands for H or OR⁸,

R⁵ and R⁶ may be identical or different and are selected from
—H, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and

R⁷ stands for H, straight-chain or branched C₁- to C₂₀-alkyl groups, a polyhydroxyl compound, such as, preferably, an ascorbic acid radical or glycosidic radicals, and

R⁸ stands for H or straight-chain or branched C₁- to C₂₀-alkyl groups, where at least 2 of the substituents R¹, R², R⁴—R⁶ are different from H or at least one substituent from R¹ and R² stands for —C(═O)—R⁷ or —C(═O)—OR⁷.

The proportion of one or more compounds selected from chromone derivatives and coumaranones in a composition is preferably from 0.001 to 5% by weight, particularly preferably from 0.01 to 2% by weight, based on the entire composition.

All compounds or components which can be used in the compositions are either known and commercially available or can be synthesised by known processes.

If the compositions are perfumes, it is preferred, in a variant of the invention, for no further assistants to be present in addition to the fragrances or perfume oils and the at least one flavonoid and typical vehicles, such as water, solvents, such as alcohols, oils and optionally emulsifiers.

If the compositions are compositions which can be applied topically, for example cosmetic or dermatological formulations, the compositions in this case comprise a cosmetically or dermatologically suitable vehicle and, depending on the desired property profile, optionally further suitable ingredients. In the case of pharmaceutical compositions, the composition in this case comprises a pharmaceutically tolerated vehicle and optionally further pharmaceutical active compounds. In the case of food supplements, a vehicle which is suitable for this purpose should be selected.

The protective action of compositions against oxidative stress or against the action of free radicals can be improved if the compositions comprise one or more antioxidants, where the person skilled in the art is presented with absolutely no difficulties in selecting antioxidants which act suitably quickly or in a delayed manner.

In a preferred embodiment, the composition is therefore a composition for the protection of body cells against oxidative stress, in particular for reducing skin ageing, characterised in that it comprises one or more antioxidants in addition to the at least one flavonoid and the at least one self-tanning substance and optionally other ingredients.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents (for example α-hydroxy-fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also compounds of the general formula A or B

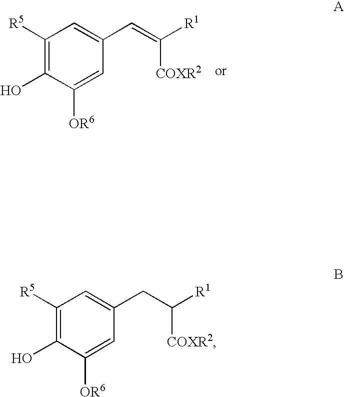

in which

R¹ can be selected from the group —C(O)CH₃, —CO₂R³, —C(O)NH₂ and —C(O)N(R⁴)₂,

X denotes O or NH,

R² denotes linear or branched alkyl having 1 to 30 C atoms,

R³ denotes linear or branched alkyl having 1 to 20 C atoms,

R⁴ in each case, independently of one another, denotes H or linear or branched alkyl having 1 to 8 C atoms,

R⁵ denotes linear or branched alkyl having 1 to 8 C atoms or linear or branched alkoxy having 1 to 8 C atoms, and

R⁶ denotes linear or branched alkyl having 1 to 8 C atoms, preferably derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST Liquid)

and/or bis(2-ethylhexyl) 2-(4-hydroxy-3,5-dimethoxybenzyl)malonate (for example RonaCare® AP).

Mixtures of antioxidants are likewise suitable for use in the cosmetic compositions according to the invention. Known and commercial mixtures are, for example, mixtures comprising, as active compounds, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-oc-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed in such compositions with the compounds according to the invention in ratios in the range from 1000:1 to 1:1000, preferably in amounts of 100:1 to 1:100.

Of the phenols which can be used in accordance with the invention, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers and I. M. C. M. Rietjens (Free Radical Biology&Medicine 2001, 31(7), 869-881), are investigating the pH dependence of the antioxidant action of hydroxyflavones. Quercetin exhibits the greatest activity amongst the structures investigated over the entire pH range.

Suitable antioxidants are furthermore compounds of the formula (III)

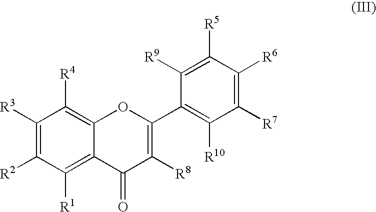

where R¹ to R¹⁰ may be identical or different and are selected from
H
OR¹¹
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ— groups, where n=1 to 3,
where all OR¹¹, independently of one another, stand for
OH
straight-chain or branched C₁- to C₂₀-alkoxy groups,
straight-chain or branched C₃- to C₂₀-alkenyloxy groups,
straight-chain or branched C₁- to C₂₀-hydroxyalkoxy groups, where the hydroxyl group(s) may be bonded to a primary or secondary carbon atom of the chain and furthermore the alkyl chain may also be interrupted by oxygen, and/or
C₃- to C₁₀-cycloalkoxy groups and/or C₃- to C₁₂-cycloalkenyloxy groups, where the rings may each also be bridged by —(CH₂)ₙ— groups, where n=1 to 3, and/or
mono- and/or oligoglycosyl radicals,
with the proviso that at least 4 radicals from R¹ to R⁷ stand for OH and that at least 2 pairs of adjacent -OH groups are present in the molecule,
or R², R⁵ and R⁶ stand for OH and the radicals R¹, R³, R⁴ and R⁷⁻¹⁰ stand for H, as described in German patent application DE-A-1 02 44 282.

The compositions to be employed may comprise vitamins as further ingredients. Preference is given to vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. In cosmetic applications, vitamins are usually added with the flavonoid-containing premixes or compositions in ranges from 0.01 to 5.0% by weight, based on the total weight. Nutrition-physiological applications depend on the respective recommended vitamin need.

Preferred compositions may also serve for sun protection and then also comprise UV filters in addition to the at least one flavonoid and the at least one self-tanning substance and optionally other ingredients.

In principle, all UV filters are suitable for a combination. Particular preference is given to UV filters whose physiological acceptability has already been demonstrated. Both for UVA and UVB filters, there are many proven substances which are known from the specialist literature, for example benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),

benzoyl- or dibenzoylmethanes, such as 1-(4-tert-butylphenyl)-3-(4-methoxy-phenyl)propane-1,3-dione (for example Eusolex® 9020) or 4-isopropyldibenzoyl-methane (for example Eusolex® 8020),

benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),

methoxycinnamic acid esters, such as octyl methoxycinnamate (for example Eusolex® 2292), isopentyl 4-methoxycinnamate, for example as a mixture of the isomers (for example Neo Heliopan® E 1000),

salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),

4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25),

phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid,

and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]hept-1-yl-methanesulfonic acid and salts thereof (for example Mexoryl® SX),
2,4,6-trianilino-(p-carbo-2′-ethylhexyl-1′-oxy)-1,3,5-triazine (for example Uvinul® T 150),
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®, drometrizoles, trisiloxanes, Mexoryl® XL),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyloxy]poly[oxy(dimethyl) [and about 6% of methyl[2-[p-[2,2-bis(ethoxycarbonyl]vinyl]phenoxy]-1-methyleneethyl] and about 1.5% of methyl[3-[p-[2,2-bis(ethoxycarbonyl)vinyl)phenoxy)propenyl) and 0.1 to 0.4% of (methylhydrogen)silylene]] (n≈60) (CAS No. 207 574-74-1),
2,2′-methylenebis(6-(2H-benzotriazol-2-yl)-4-(1,1,3,3-tetramethylbutyl)phenol) (CAS No.103 597-45-1),
2,2′-(1,4-phenylene)bis(1H-benzimidazole-4,6-disulfonic acid, monosodium salt) (CAS No.180 898-37-7) and
2,4-bis{[4-(2-ethylhexyloxy)-2-hydroxy]phenyl}-6-(4-methoxyphenyl)-1,3,5-tri-azine (CAS No. 103 597-45-, 187 393-00-6).

Further suitable UV filters are also methoxyflavones corresponding to German patent application DE-A-10232595.

Organic UV filters are generally incorporated into formulations in an amount of 0.5 to 20 percent by weight, preferably 1-15%.

In order to ensure optimised UV protection, it is furthermore preferred for compositions having light-protection properties also to comprise inorganic UV filters. Conceivable inorganic UV filters are those from the group of the titanium dioxides, such as, for example, coated titanium dioxide (for example Eusolex® T-2000, Eusolex® T-AQUA, Eusolex® T-AVO), zinc oxides (for example Sachtotec®), iron oxides or also cerium oxides. These inorganic UV filters are generally incorporated into cosmetic compositions in an amount of 0.5 to 20 percent by weight, preferably 2-10%.

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl meth-oxycinnamate, 3,3,5-trimethylcyclohexyl salicylate, 2-ethylhexyl 4-(dimethyl-amino)benzoate, 2-ethylhexyl 2-cyano-3,3-diphenylacrylate, 2-phenylbenzimidazole-5-sulfonic acid and potassium, sodium and triethanolamine salts thereof.

The protective action against damaging effects of UV radiation can be optimised by combining one or more of the said compounds having a UV-filter action.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin are repeatedly discussed in the literature. The encapsulation of the corresponding substances which is proposed here suppresses this effect.
In general, encapsulation of individual UV filters or other ingredients enables formulation problems caused by the interaction of individual composition constituents with one another, such as crystallisation processes, precipitation and agglomerate formation, to be avoided since the interaction is suppressed.

It is therefore preferred for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active compound (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules particularly preferably to be employed have walls which can be obtained by a sol-gel process, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules in compositions to be employed in accordance with the invention are preferably present in amounts which ensure that the encapsulated UV filters are present in the composition in the above-indicated amounts.

Particularly preferred active compounds, in particular for skin-care compositions, are, for example, also so-called compatible solutes. These are substances which are involved in the osmoregulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and the respective precursors thereof. For the purposes of German patent application DE-A-10133202, osmolytes are taken to mean, in particular, substances from the group of the polyols, such as, for example, myoinositol, mannitol or sorbitol and/or one or more of the osmolytically active substances mentioned below: taurine, choline, betaine, phosphorylcholine, glycerophosphorylcholines, glutamine, glycine, α-alanine, glutamate, aspartate, proline, and taurine. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

In the self-tanning compositions described, compatible solutes employed are preferably substances selected from the group consisting of pyrimidinecarboxylic acids (such as ectoine and hydroxyectoine), proline, betaine, glutamine, cyclic di-phosphoglycerate, N-acetylornithine, trimethylamine N-oxide, di-myo-inositol phosphate (DIP), cyclic 2,3-diphosphoglycerate (cDPG), 1,1-diglycerol phosphate (DGP), β-mannosyl glycerate (firoin), β-mannosylglyceramide (firoin A) or/and dimannosyl diinositol phosphate (DMIP) or an optical isomer, derivative, for example an acid, a salt or ester of these compounds, or combinations thereof.

Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoine, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoine, can be used as protectant in dried yeast and bacteria cells. Pharmaceutical products, such as non-glycosylated, pharmaceutically active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European patent application EP-A-0 671 1 61 describes, in particular, that ectoine and hydroxyectoine are employed in cosmetic compositions, such as powders, soaps, surfactant-containing cleansing products, lipsticks, rouge, make-up, care creams and sunscreen preparations.

Preference is given here to the use of a pyrimidinecarboxylic acid of the following formula

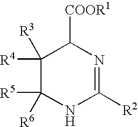

in which R¹ is a radical H or C1-8-alkyl, R² is a radical H or C1-4-alkyl, and R³, R⁴, R⁵ and R⁶ are each, independently of one another, a radical from the group H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid). The compositions to be employed in accordance with the invention preferably comprise pyrimidinecarboxylic acids of this type in amounts of up to 15% by weight.

It is particularly preferred here for the compatible solutes to be selected from dimyo-inositol phosphate (DIP), cyclic 2,3-diphosphoglycerate (cDPG), 1,1-diglycerol phosphate (DGP), β-mannosyl glycerate (firoin), β-mannosylglyceramide (firoin A) or/and dimannosyl diinositol phosphate (DMIP), ectoine, hydroxyectoine or mixtures thereof.

Of the aryl oximes likewise preferably employed, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and skin appendages. Compositions according to the invention which additionally comprise an aryl oxime, preferably 2-hydroxy-5-methyllauro-phenone oxime, exhibit anti-inflammatory suitability. The compositions here preferably comprise 0.01 to 10% by weight of the aryl oxime, it being particularly preferred for the composition to comprise 0.05 to 5% by weight of aryl oxime.

The at least one flavonoid can be incorporated into the compositions in a conventional manner, for example by mixing. The present invention furthermore relates to a corresponding preparation process.

As use form of the compositions to be employed, mention may be made, for example, of: solutions, suspensions, emulsions, PIT emulsions, pastes, ointments, gels, creams, lotions, powders, soaps, surfactant-containing cleansing preparations, oils, aerosols and sprays. Preferred use forms are also shampoos, sun baths and shower baths, which are also known as spray tanning, airbrush tanning or sun showers from commercial self-tanning studios.

Preferred assistants originate from the group of the preservatives, antioxidants, stabilisers, solubilisers, vitamins, colorants and odour improvers.

Ointments, pastes, creams and gels may comprise the customary vehicles, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary readily volatile, liquefied propellants, for example chlorofluorocarbons, propane/butane or dimethyl ether. Compressed air can also advantageously be used.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary vehicles, such as liquid diluents, for example water, ethanol or propylene glycol, suspending media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic use forms are also lip-care sticks, powder, emulsion and wax make-up, and sunscreen, pre-sun and after-sun preparations.

The preferred composition forms also include, in particular, emulsions.

Emulsions are advantageous and comprise, for example, the said fats, oils, waxes and other lipids, as well as water and an emulsifier, as usually used for a composition of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic lipids, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of the esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, from the group of the esters of aromatic carboxylic acids and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group of the branched and unbranched hydrocarbons and waxes, silicone oils, dialkyl ethers, the group of the saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24 C atoms, in particular 12-18 C atoms. The fatty acid triglycerides may advantageously be selected, for example, from the group of the synthetic, semisynthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The aqueous phase of the compositions to be employed optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions are advantageous and comprise, for example, the said fats, oils, waxes and other lipids, as well as water and an emulsifier, as usually used for a formulation of this type.

In a preferred embodiment, the compositions to be employed comprise hydrophilic surfactants. The hydrophilic surfactants are preferably selected from the group of the alkylglucosides, acyl lactylates, betaines and coconut amphoacetates.

It is likewise advantageous to employ natural or synthetic raw materials and assistants or mixtures which are distinguished by an effective content of the active compounds used in accordance with the invention, for example Plantaren® 1200 (Henkel KGaA), Oramix® NS 10 (Seppic).

The composition may furthermore comprise adjuvants which are usually used in compositions of this type, such as, for example, thickeners, softeners, humectants, interface-active agents, emulsifiers, preservatives, antifoam agents, waxes, lanolin, propellants, dyes and/or pigments, which colour the composition itself or the skin, and other ingredients usually used in cosmetics.

The composition may also be in the form of an alcoholic gel which comprises one or more lower alcohols or polyols, such as ethanol, propylene glycol or glycerol, and a thickener, such as siliceous earth. The oily/alcoholic gels additionally comprise natural or synthetic oil or wax.

The solid sticks consist of natural or synthetic waxes and oils, fatty alcohols, fatty acids, fatty acid esters, lanolin and other lipids.

If a composition is formulated as an aerosol, the usual propellants, such as alkanes, fluoroalkanes and chlorofluoroalkanes, preferably alkanes, are generally used.

The compositions to be employed can be prepared with the aid of techniques which are well known to the person skilled in the art.

Even without further comments, it is assumed that a person skilled in the art will be able to utilise the above description in the broadest scope. The preferred embodiments should therefore merely be regarded as descriptive disclosure which is absolutely not limiting in any way. The complete disclosure content of all applications and publications mentioned above and below is incorporated into this application by way of reference.

The examples of the subject-matter according to the invention that are given below serve merely for explanation and absolutely do not restrict the present invention in any way. In addition, the invention described can be carried out throughout the entire range claimed. All compounds or components which can be used in the compositions are either known and commercially available or can be synthesised by known methods. The INCI names of the raw materials used are indicated (the INCI names are by definition indicated in English).

### EXAMPLE 1

### Stabilisation of Vanillin by Troxerutin (Model for an Aqueous/Ethanolic Phase of an Emulsion System)

Procedure: 10 mg of vanillin are dissolved in water/ethanol (50/50) in 10 ml volumetric flasks with (Experiment A) or without (Experiment B) 100 mg of troxerutin and made up to exactly 10 ml. The mixture is subsequently transferred into a 20 ml screw-cap glass bottle (HPLC sample: 100 ml made up to 1000 ml) and stirred vigorously while open for 96 hours. The samples are rinsed back into the volumetric flasks, rinsed with ethanol and made up to exactly 10 ml. The HPLC evaluation is carried out in accordance with the peak areas (again 100 ml sample).

A (troxerutin + vanillin)

HPLC-2

| t [h] | Area | Vanillin* |
|---|---|---|
| 0 | 862860 | 100% |
| 96 | 840319 | 97% |

B (vanillin)

HPLC-2

| t [h] | Area | Vanillin* |
|---|---|---|
| 0 | 897239 | 100% |
| 96 | 689849 | 77% |

After performance of the experiment, 77% by weight of the vanillin originally employed are recovered without stabilisation, while 97% by weight are recovered in the presence of troxerutin.

Compositions

Illustrative formulations of cosmetic compositions which comprise a flavonoid, for example rutin or troxerutin, and a self-tanning substance, for example DHA, are indicated below. In addition, the INCI names of the commercially available compounds are indicated. The DHA shower solutions are prepared by weighing out all raw materials and stirring until homogeneous.

### EXAMPLE 2

### DHA Shower Solution

| INCI | [% by weight] |
|---|---|
| Aqua (Water) | 75.05 |
| Dihydroxyacetone | 8.00 |
| Rutin | 0.75 |
| Ectoine | 0.30 |
| Propylene glycol | 4.50 |
| Glycerin | 2.00 |
| Ethoxydiglycol | 5.00 |
| Dimethyl isosorbide | 2.00 |
| Polysorbate 80 | 0.50 |
| Propylene glycol, walnut extract | 1.50 |
| Caramel | 0.10 |
| Perfume | 0.30 |

### EXAMPLE 3

### DHA Shower Solution

Composition analogous to Example 2, only with 0.75% of troxerutin employed instead of rutin.

### EXAMPLE 4

### Self-Tanning Cream with Flavonoid (O/W)

The self-tanning cream is prepared by heating phase A (consisting of glyceryl stearate, stearyl alcohol, cetearyl alcohol, cetearyl ethylhexanoate, caprylic triglyceride, stearoxydimethicone, dimethicone, tocopheryl acetate and propylparaben) and phase B (consisting of propylene glycol, methylparaben and water). Phase B is slowly stirred into phase A, and the mixture is homogenised. The mixture is cooled with stirring. The rutin is dissolved in the water before the DHA. Phase C (consisting of DHA, rutin and water) is added at 40° C.

| INCI | [% by weight] |
|---|---|
| Aqua (water) | 69.00 |
| Dihydroxyacetone | 2.00 |
| Rutin | 1.00 |
| Methylparaben | 0.15 |
| Propylene glycol | 3.00 |
| Glyceryl stearate, stearyl alcohol | 8.00 |
| CETETH-20, STEARETH-25 | |
| Cetearyl alcohol | 1.50 |
| Cetearyl ethylhexanoate | 6.50 |
| Caprylic/Capric triglyceride | 6.50 |
| Stearoxy dimethicone | 1.20 |
| Dimethicone | 0.50 |
| Tocopheryl acetate | 0.50 |
| Propylparaben | 0.05 |
| Perfume | 0.10 |

### EXAMPLE 5

### Self-Tanning Cream with Flavonoid (O/W)

Composition analogous to Example 4, only with 1% of troxerutin employed instead of rutin.

### EXAMPLE 6

### Self-Tanning Cream with Flavonoid (O/W)

| INCI | [% by weight] |
|---|---|
| Aqua (water) | to 100 |
| Dihydroxyacetone | 0.75 |
| Troxerutin | 0.5 |
| Methylparaben | 0.15 |
| Propylene glycol | 3.00 |
| Glyceryl stearate, stearyl alcohol | 8.00 |
| CETETH-20, STEARETH-25 | |
| Cetearyl alcohol | 1.50 |
| Cetearyl ethylhexanoate | 6.50 |
| Caprylic/Capric triglyceride | 6.50 |
| Stearoxy dimethicone | 1.20 |
| Dimethicone | 0.50 |
| Tocopheryl acetate | 0.50 |
| Propylparaben | 0.05 |
| Perfume | 0.10 |

### COMPARATIVE EXAMPLE 6C

### Cream According to Example 6, but Without Troxerutin

### EXAMPLE 7

### Odour Change on Storage

The creams according to Example 6 or 6C are stored for 1 year at room temperature in sealed crucibles. After the year, the two formulations are additionally prepared freshly. The sensory test shows that 6C has changed significantly in odour compared with the fresh composition, the original odour is substantially lost, while the odour of Example 6 hardly changes in the year.

### EXAMPLE 8

### Odour on the Skin

The creams according to Example 6 or 6C are freshly prepared and applied to the human skin, and the odour immediately after application and a few hours after application is assessed sensorily.

Immediately after application, the odour of the skin areas treated with the creams is found to be pleasant in the case of both creams. A few hours later, only the odour of the skin areas treated with Example 6 are described as pleasant—slightly flowery or vanilla-like—by the test subjects, while the odour of the skin areas treated with Comparative Example 6C is described by the test subjects as unpleasant or similar to the odour of lovage.

### EXAMPLE 9

### DHA Shower Lotion

| | INCI | | | | | |
|---|---|---|---|---|---|---|
| | WY-02-01 % | WY-02-02 % | WY-02-03 % | WY-02-04 % | WY-02-05 % | WY-02-06 % |
| Dihydroxyacetone | 8.00 | 10.00 | 8.00 | 10.00 | | |
| Dihydroxyacetone, | | | | | 8.00 | 10.00 |
| Sodium Metabisulfite, | | | | | | |
| Magnesium stearate | | | | | | |
| Sodium Metabisulfite | 0.80 | 1.00 | 0.80 | 1.00 | | |
| Troxerutin | 1.00 | 2.00 | 1.00 | 2.00 | 1.00 | 2.00 |
| Colorona ® Bronze | | | 2.00 | 3.00 | | |
| Erythrulose | | | | | | |
| Ectoine | 0.50 | 0.50 | 0.50 | 0.50 | 0.50 | 0.50 |
| Propylene Glycol | 6.00 | 6.00 | 6.00 | 6.00 | 6.00 | 6.00 |
| Ethoxydiglycol | 5.00 | 5.00 | 5.00 | 5.00 | 5.00 | 5.00 |
| Glycerin | 4.00 | 4.00 | 4.00 | 4.00 | 4.00 | 4.00 |
| Dimethyl Isosorbide | 2.00 | 2.00 | 2.00 | 2.00 | 2.00 | 2.00 |
| Polysorbate 80 | 1.60 | 1.60 | 1.60 | 1.60 | 1.60 | 1.60 |
| Aloe Barbadensis | 0.30 | 0.30 | 0.30 | 0.30 | 0.30 | 0.30 |
| Colorants | q.s. | q.s. | q.s. | q.s. | q.s. | q.s. |
| Preservative | q.s. | q.s. | q.s. | q.s. | q.s. | q.s. |
| Aqua (water) | 70.80 | 67.60 | 68.80 | 64.60 | 71.60 | 68.60 |
| to | 100.00 | 100.00 | 100.00 | 100.00 | 100.00 | 100.00 |
| | INCI | | | | | |
| | WY-02-07 % | WY-02-08 % | WY-02-09 % | WY-02-10 % | WY-02-11 % | WY-02-12 % |
| Dihydroxyacetone | | | 8.00 | 10.00 | 10.00 | |
| Dihydroxyacetone, | 8.00 | 10.00 | | | | 8.00 |
| Sodium Metabisulfite, | | | | | | |
| Magnesium stearate | | | | | | |
| Sodium Metabisulfite | | | 0.80 | 1.00 | 1.00 | |
| Troxerutin | 1.00 | 2.00 | 1.00 | 2.00 | 2.00 | 1.00 |
| Colorona ® Bronze | 2.00 | 3.00 | | | 3.00 | |
| Erythrulose | | | 3.00 | 3.50 | 3.50 | 3.00 |
| Ectoine | 0.50 | 0.50 | 0.50 | 0.50 | 0.50 | 0.50 |
| Propylene Glycol | 6.00 | 6.00 | 6.00 | 6.00 | 6.00 | 6.00 |
| Ethoxydiglycol | 5.00 | 5.00 | 5.00 | 5.00 | 5.00 | 5.00 |
| Glycerin | 4.00 | 4.00 | 4.00 | 4.00 | 4.00 | 4.00 |
| Dimethyl Isosorbide | 2.00 | 2.00 | 2.00 | 2.00 | 2.00 | 2.00 |
| Polysorbate 80 | 1.60 | 1.60 | 1.60 | 1.60 | 1.60 | 1.60 |
| Aloe Barbadensis | 0.30 | 0.30 | 0.30 | 0.30 | 0.30 | 0.30 |
| Colorants | q.s. | q.s. | q.s. | q.s. | q.s. | q.s. |
| Preservative | q.s. | q.s. | q.s. | q.s. | q.s. | q.s. |
| Aqua (water) | 69.60 | 65.60 | 67.80 | 64.10 | 61.10 | 68.60 |
| Complete | 100.00 | 100.00 | 100.00 | 100.00 | 100.00 | 100.00 |
| | INCI | | | | | |
| | WY-02-13 % | WY-02-14 % | WY-02-15 % | WY-02-16 % | WY-02-17 % | WY-02-18 % |
| Dihydroxyacetone | | | 8.00 | 10.00 | 10.00 | |
| Dihydroxyacetone, | 10.00 | 10.00 | | | | 8.00 |
| Sodium Metabisulfite, | | | | | | |
| Magnesium stearate | | | | | | |
| Sodium Metabisulfite | | | | 1.00 | 1.00 | |
| Troxerutin | 2.00 | 2.00 | 1.00 | | | |
| Rutin sulfate | | | | 2.00 | 2.00 | 1.00 |
| Colorona ® Bronze | | 3.00 | | | 3.00 | |
| Erythrulose | 3.50 | 3.50 | 3.00 | 3.50 | 3.50 | 3.00 |
| Ectoine | 0.50 | 0.50 | 0.50 | 0.50 | 0.50 | 0.50 |
| Propylene Glycol | 6.00 | 6.00 | 6.00 | 6.00 | 6.00 | 6.00 |
| Ethoxydiglycol | 5.00 | 5.00 | 5.00 | 5.00 | 5.00 | 5.00 |
| Glycerin | 4.00 | 4.00 | 4.00 | 4.00 | 4.00 | 4.00 |
| Dimethyl Isosorbide | 2.00 | 2.00 | 2.80 | 2.00 | 2.00 | 2.00 |
| Polysorbate 80 | 1.60 | 1.60 | 1.60 | 1.60 | 1.60 | 1.60 |
| Aloe Barbadensis | 0.30 | 0.30 | 0.30 | 0.30 | 0.30 | 0.30 |
| Colorants | q.s. | q.s. | q.s. | q.s. | q.s. | q.s. |
| Preservative | q.s. | q.s. | q.s. | q.s. | q.s. | q.s. |
| Aqua (water) | 65.10 | 62.10 | 66.80 | 64.10 | 61.10 | 68.60 |
| Complete | 100.00 | 100.00 | 100.00 | 100.00 | 100.00 | 100.00 |

Process: All raw materials are stirred to homogeneity

| | WY-02-28 | WY-02-29 | WY-02-30 | WY-02-31 |
|---|---|---|---|---|
| INCI | % | % | % | % |
| Dihydroxyacetone | 8.00 | 10.00 | 8.00 | 10.00 |
| Troxerutin | 2.00 | 3.00 | 2.00 | 3.00 |
| Colorona ® Bronze | 3.00 | 5.00 | 3.00 | 5.00 |
| Erythrulose | | | 3.00 | 5.00 |
| Ectoine | 0.50 | 0.50 | 0.50 | 0.50 |
| Propylene Glycol | 6.00 | 6.00 | 6.00 | 6.00 |
| Ethoxydiglycol | 5.00 | 5.00 | 5.00 | 5.00 |
| Glycerin | 4.00 | 4.00 | 4.00 | 4.00 |
| Dimethyl Isosorbide | 2.00 | 2.00 | 2.00 | 2.00 |
| Polysorbate 80 | 1.60 | 1.60 | 1.60 | 1.60 |
| Aloe Barbadensis | 0.30 | 0.30 | 0.30 | 0.30 |
| Colorants | q.s. | q.s. | q.s. | q.s. |
| Preservative | q.s. | q.s. | q.s. | q.s. |
| Aqua (water) | 67.60 | 62.60 | 64.60 | 57.60 |
| to | 100.00 | 100.00 | 100.00 | 100.00 |

Process: All raw materials are stirred to homogeneity

### EXAMPLE 10

### DHA Shower Lotion

| | INCI | | | | | |
|---|---|---|---|---|---|---|
| | WY-01-01 % | WY-01-02 % | WY-01-03 % | WY-01-04 % | WY-01-05 % | WY-01-06 % |
| Dihydroxyacetone | 8.00 | 10.00 | 8.00 | 10.00 | | |
| Dihydroxyacetone, | | | | | 8.00 | 10.00 |
| Sodium Metabisulfite, | | | | | | |
| Magnesium stearate | | | | | | |
| Sodium Metabisulfite | 0.80 | 1.00 | 0.80 | 1.00 | | |
| Troxerutin | 1.00 | 2.00 | 1.00 | 2.00 | 1.00 | 2.00 |
| Colorona ® Bronze | | | 2.00 | 3.00 | | |
| Erythrulose | | | | | | |
| Ectoine | 0.50 | 0.50 | 0.50 | 0.50 | 0.50 | 0.50 |
| Propylene Glycol | 4.50 | 4.50 | 4.50 | 4.50 | 4.50 | 4.50 |
| Glycerin | 4.00 | 4.00 | 4.00 | 4.00 | 4.00 | 4.00 |
| Ethoxydiglycol | 3.00 | 3.00 | 3.00 | 3.00 | 3.00 | 3.00 |
| Dimethyl Isosorbide | 2.00 | 2.00 | 2.00 | 2.00 | 2.00 | 2.00 |
| Polysorbate 80 | 0.50 | 0.50 | 0.50 | 0.50 | 0.50 | 0.50 |
| Walnut Extract, | 1.50 | 1.50 | 1.50 | 1.50 | 1.50 | 1.50 |
| Propylene Glycol | | | | | | |
| Caramel | 0.10 | 0.10 | 0.10 | 0.10 | 0.10 | 0.10 |
| Perfume | 0.30 | 0.30 | 0.30 | 0.30 | 0.30 | 0.30 |
| Phenoxyethanol, Butyl- | 0.80 | 0.80 | 0.80 | 0.80 | 0.80 | 0.80 |
| paraben, Ethylparaben, | | | | | | |
| Propylparaben, Methyl- | | | | | | |
| paraben | | | | | | |
| Aqua (water) | 73.00 | 69.80 | 71.00 | 66.80 | 73.80 | 68.60 |
| Complete | 100.00 | 100.00 | 100.00 | 100.00 | 100.00 | 100.00 |

Process: All raw materials are stirred to homogeneity

| | INCI | | | | | |
|---|---|---|---|---|---|---|
| | WY-01-07 % | WY-01-08 % | WY-01-09 % | WY-01-10 % | WY-01-11 % | WY-01-12 % |
| Dihydroxyacetone | | | 8.00 | 10.00 | 10.00 | |
| Dihydroxyacetone, | 8.00 | 10.00 | | | | 8.00 |
| Sodium Metabisulfite, | | | | | | |
| Magnesium stearate | | | | | | |
| Sodium Metabisulfite | | | 0.80 | 1.00 | 1.00 | |
| Troxerutin | 1.00 | 2.00 | 1.00 | 2.00 | 2.00 | 1.00 |
| Colorona ® Bronze | 2.00 | 3.00 | | | 3.00 | |
| Erythrulose | | | 3.00 | 3.50 | 3.50 | 3.00 |
| Ectoine | 0.50 | 0.50 | 0.50 | 0.50 | 0.50 | 0.50 |
| Propylene Glycol | 4.50 | 4.50 | 4.50 | 4.50 | 4.50 | 4.50 |
| Glycerin | 4.00 | 4.00 | 4.00 | 4.00 | 4.00 | 4.00 |
| Ethoxydiglycol | 3.00 | 3.00 | 3.00 | 3.00 | 3.00 | 3.00 |
| Dimethyl Isosorbide | 2.00 | 2.00 | 2.00 | 2.00 | 2.00 | 2.00 |
| Polysorbate 80 | 0.50 | 0.50 | 0.50 | 0.50 | 0.50 | 0.50 |
| Walnut Extract, | 1.50 | 1.50 | 1.50 | 1.50 | 1.50 | 1.50 |
| Propylene Glycol | | | | | | |
| Caramel | 0.10 | 0.10 | 0.10 | 0.10 | 0.10 | 0.10 |
| Perfume | 0.30 | 0.30 | 0.30 | 0.30 | 0.30 | 0.30 |
| Phenoxyethanol, Butyl- | 0.80 | 0.80 | 0.80 | 0.80 | 0.80 | 0.80 |
| paraben, Ethylparaben, | | | | | | |
| Propylparaben, Methyl- | | | | | | |
| paraben | | | | | | |
| Aqua (water) | 71.80 | 67.80 | 70.00 | 66.30 | 63.30 | 70.80 |
| Complete | 100.00 | 100.00 | 100.00 | 100.00 | 100.00 | 100.00 |

Process: All raw materials are stirred to homogeneity

| | WY-01-13 | WY-01-14 | WY-01-15 | WY-01-16 |
|---|---|---|---|---|
| INCI | % | % | % | % |
| Dihydroxyacetone | 8.00 | 10.00 | 8.00 | 10.00 |
| Troxerutin | 2.00 | 3.00 | 2.00 | 3.00 |
| Colorona ® Bronze | 3.00 | 5.00 | 3.00 | 5.00 |
| Erythrulose | | | 3.00 | 5.00 |
| Ectoine | 0.50 | 0.50 | 0.50 | 0.50 |
| Propylene Glycol | 4.50 | 4.50 | 4.50 | 4.50 |
| Glycerin | 4.00 | 4.00 | 4.00 | 4.00 |
| Ethoxydiglycol | 3.00 | 3.00 | 3.00 | 3.00 |
| Dimethyl Isosorbide | 2.00 | 2.00 | 2.00 | 2.00 |
| Polysorbate 80 | 0.50 | 0.50 | 0.50 | 0.50 |
| Walnut Extract, | 1.50 | 1.50 | 1.50 | 1.50 |
| Propylene Glycol | | | | |
| Caramel | 0.10 | 0.10 | 0.10 | 0.10 |
| Perfume | 0.30 | 0.30 | 0.30 | 0.30 |
| Phenoxyethanol, Butyl- | 0.80 | 0.80 | 0.80 | 0.80 |
| araben, Ethylparaben, | | | | |
| Propylparaben, Methyl- | | | | |
| araben | | | | |
| Aqua (water) | 71.00 | 66.80 | 73.80 | 70.80 |
| Complete | 101.20 | 102.00 | 107.00 | 111.00 |

Process: All raw materials are stirred to homogeneity

### EXAMPLE 11

### Hydrogel Comprising DHA

| | INCI | | | | | |
|---|---|---|---|---|---|---|
| | SF-40-16 % | SF-40-17 % | SF-40-18 % | SF-40-19 % | SF-40-20 % | SF-40-21 % |
| Phase A | | | | | | |
| Dihydroxyacetone | 1.00 | 2.00 | 1.00 | 2.00 | | |
| Dihydroxyacetone, | | | | | 1.00 | 2.00 |
| Sodium Metabisulfite, | | | | | | |
| Magnesium stearate | | | | | | |
| Sodium Metabisulfite | 0.10 | 0.20 | 0.10 | 0.20 | | |
| Troxerutin | 0.50 | 1.00 | 0.50 | 1.00 | 0.50 | 1.00 |
| Colorona ® Bronze | | | 1.00 | 2.00 | | |
| Erythrulose | | | | | | |
| Ectoine | 0.30 | 0.30 | 0.30 | 0.30 | 0.30 | 0.30 |
| Propylene Glycol | 2.50 | 2.50 | 2.50 | 2.50 | 2.50 | 2.50 |
| Sorbitol | 2.50 | 2.50 | 2.50 | 2.50 | 2.50 | 2.50 |
| Methylparaben | 0.20 | 0.20 | 0.20 | 0.20 | 0.20 | 0.20 |
| Aqua (water) | 31.40 | 29.80 | 30.40 | 27.80 | 31.50 | 30.0 |
| Phase B | | | | | | |
| Aqua (water) | 60.00 | 60.00 | 60.00 | 60.00 | 60.00 | 60.00 |
| Hydroxyethylcellulose | 1.50 | 1.50 | 1.50 | 1.50 | 1.50 | 1.50 |
| Complete | 100.00 | 100.00 | 100.00 | 100.00 | 100.00 | 100.00 |
| | INCI | | | | | |
| | SF-40-22 % | SF-40-23 % | SF-40-24 % | SF-40-25 % | SF-40-26 % | SF-40-27 % |
| Phase A | | | | | | |
| Dihydroxyacetone | | | 5.00 | | 5.00 | |
| Dihydroxyacetone, | 1.00 | 2.00 | | 2.00 | | 5.00 |
| Sodium Metabisulfite, | | | | | | |
| Magnesium stearate | | | | | | |
| Sodium Metabisulfite | | | 0.50 | | 0.50 | |
| Troxerutin | 0.50 | 1.00 | 2.50 | 2.50 | 2.50 | 2.50 |
| Colorona ® Bronze | 1.00 | 2.00 | | | 5.00 | 5.00 |
| Erythrulose | | | | | | |
| Ectoine | 0.30 | 0.30 | 0.30 | 0.30 | 0.30 | 0.30 |
| Propylene Glycol | 2.50 | 2.50 | 2.50 | 2.50 | 2.50 | 2.50 |
| Sorbitol | 2.50 | 2.50 | 2.50 | 2.50 | 2.50 | 2.50 |
| Methylparaben | 0.20 | 0.20 | 0.20 | 0.20 | 0.20 | 0.20 |
| Aqua (water) | 30.50 | 28.00 | 25.00 | 25.50 | 20.00 | 20.50 |
| Phase B | | | | | | |
| Aqua (water) | 60.00 | 60.00 | 60.00 | 60.00 | 60.00 | 60.00 |
| Hydroxyethylcellulose | 1.50 | 1.50 | 1.50 | 1.50 | 1.50 | 1.50 |
| Complete | 100.00 | 100.00 | 100.00 | 100.00 | 100.00 | 100.00 |
| | INCI | | | | | |
| | SF-40-28 % | SF-40-29 % | SF-40-30 % | SF-40-31 % | SF-40-32 % | SF-40-33 % |
| Phase A | | | | | | |
| Dihydroxyacetone | 1.00 | 2.00 | | | 5.00 | 5.00 |
| Dihydroxyacetone, | | | 1.00 | 2.00 | | |
| Sodium Metabisulfite, | | | | | | |
| Magnesium stearate | | | | | | |
| Sodium Metabisulfite | 0.10 | 0.20 | | | 0.50 | 0.50 |
| Troxerutin | 0.50 | 1.00 | 0.50 | 1.00 | 2.50 | 2.50 |
| Colorona ® Bronze | | | | | | 5.00 |
| Erythrulose | 2.00 | 2.00 | 2.00 | 2.00 | 2.00 | 2.00 |
| Ectoine | 0.30 | 0.30 | 0.30 | 0.30 | 0.30 | 0.30 |
| Propylene Glycol | 2.50 | 2.50 | 2.50 | 2.50 | 2.50 | 2.50 |
| Sorbitol | 2.50 | 2.50 | 2.50 | 2.50 | 2.50 | 2.50 |
| Methylparaben | 0.20 | 0.20 | 0.20 | 0.20 | 0.20 | 0.20 |
| Aqua (water) | 29.40 | 27.80 | 29.50 | 28.00 | 23.00 | 18.00 |
| Phase B | | | | | | |
| Aqua (water) | 60.00 | 60.00 | 60.00 | 60.00 | 60.00 | 60.00 |
| Hydroxyethylcellulose | 1.50 | 1.50 | 1.50 | 1.50 | 1.50 | 1.50 |
| Complete | 100.00 | 100.00 | 100.00 | 100.00 | 100.00 | 100.00 |
| | INCI | | | | | |
| | SF-40-34 % | SF-40-35 % | SF-40-36 % | SF-40-37 % | SF-40-38 % | SF-40-39 % |
| Phase A | | | | | | |
| Dihydroxyacetone | | | 1.00 | 2.00 | 5.00 | 5.00 |
| Dihydroxyacetone, | 5.00 | 5.00 | | | | |
| Sodium Metabisulfite, | | | | | | |
| Magnesium stearate | | | | | | |
| Sodium Metabisulfite | | | | | | |
| Troxerutin | 2.50 | 2.50 | 0.50 | 1.00 | 2.50 | 0.50 |
| Colorona ® Bronze | | 5.00 | 1.00 | 2.00 | 5.00 | 1.00 |
| Erythrulose | 2.00 | 2.00 | | | | 0.50 |
| Ectoine | 0.30 | 0.30 | 0.30 | 0.30 | 0.30 | 0.30 |
| Propylene Glycol | 2.50 | 2.50 | 2.50 | 2.50 | 2.50 | 2.50 |
| Sorbitol | 2.50 | 2.50 | 2.50 | 2.50 | 2.50 | 2.50 |
| Methylparaben | 0.20 | 0.20 | 0.20 | 0.20 | 0.20 | 0.20 |
| Aqua (water) | 23.50 | 18.50 | 30.50 | 28.00 | 20.50 | 30.00 |
| Phase B | | | | | | |
| Aqua (water) | 60.00 | 60.00 | 60.00 | 60.00 | 60.00 | 60.00 |
| Hydroxyethylcellulose | 1.50 | 1.50 | 1.50 | 1.50 | 1.50 | 1.50 |
| Complete | 100.00 | 100.00 | 100.00 | 100.00 | 100.00 | 100.00 |

INCI

| | SF-40-40 % | SF-40-41 % |
|---|---|---|
| Phase A | | |
| Dihydroxyacetone | 2.00 | 5.00 |
| Dihydroxyacetone, | | |
| Sodium Metabisulfite, | | |
| Magnesium stearate | | |
| Sodium Metabisulfite | | |
| Troxerutin | 1.00 | 2.50 |
| Colorona ® Bronze | 2.00 | 5.00 |
| Erythrulose | 1.00 | 2.50 |
| Ectoine | 0.30 | 0.30 |
| Propylene Glycol | 2.50 | 2.50 |
| Sorbitol | 2.50 | 2.50 |
| Methylparaben | 0.20 | 0.20 |
| Aqua (water) | 27.00 | 18.00 |
| Phase B | | |
| Aqua (water) | 60.00 | 60.00 |
| Hydroxyethylcellulose | 1.50 | 1.50 |
| Complete | 100.00 | 100.00 |

Process:

Hydroxyethylcellulose is added to the water of phase B with vigorous stirring. The addition must be carried out at a rate which allows the particles to separate and to wet their surface, but it must be ensured that the viscosity is minimised.

Storage at room temperature is recommended.

### EXAMPLE 12

### W/O Lotion with Troxerutin

| | INCI | | | | | | |
|---|---|---|---|---|---|---|---|
| | ST-16-42 % | ST-16-43 % | ST-16-44 % | ST-16-45 % | ST-16-46 % | ST-16-47 % | ST-16-48 % |
| Phase A | | | | | | | |
| Bis-PEG/PPG-14/14 | 1.50 | 1.50 | 1.50 | 1.50 | 1.50 | 1.50 | 1.50 |
| Dimethicone, | | | | | | | |
| Cyclopentasiloxane | | | | | | | |
| Cetyl PEG/PPG-10/1 | 1.30 | 1.30 | 1.30 | 1.30 | 1.30 | 1.30 | 1.30 |
| Dimethicone | | | | | | | |
| Cyclomethicone | 13.00 | 13.00 | 13.00 | 13.00 | 13.00 | 13.00 | 13.00 |
| Ethylhexyl Palmitate | 2.00 | 2.00 | 2.00 | 2.00 | 2.00 | 2.00 | 2.00 |
| Cyclopentasiloxane, | 5.00 | 5.00 | 5.00 | 5.00 | 5.00 | 5.00 | 5.00 |
| Dimethicone/Vinyl- | | | | | | | |
| dimethicone, | | | | | | | |
| Crosspolymer | | | | | | | |
| Diethylhexyl | 5.00 | 5.00 | 5.00 | 5.00 | 5.00 | 5.00 | 5.00 |
| Carbonate | | | | | | | |
| Perfume | 0.30 | 0.30 | 0.30 | 0.30 | 0.30 | 0.30 | 0.30 |
| Phase B | | | | | | | |
| Dihydroxyacetone | 1.00 | 2.00 | 1.00 | 2.00 | | | |
| Dihydroxyacetone, | | | | | 1.00 | 2.00 | 1.00 |
| Sodium Metabisulfite, | | | | | | | |
| Magnesium stearate | | | | | | | |
| Sodium Metabisulfite | 0.10 | 0.20 | 0.10 | 0.20 | | | |
| Troxerutin | 0.50 | 1.00 | 0.50 | 1.00 | 0.50 | 1.00 | 0.50 |
| Colorona ® Bronze | | | 1.00 | 2.00 | | | |
| Erythrulose | | | | | | | |
| Ectoine | 0.30 | 0.30 | 0.30 | 0.30 | 0.30 | 0.30 | 0.30 |
| Propylene Glycol | 18.00 | 18.00 | 18.00 | 18.00 | 18.00 | 18.00 | 18.00 |
| Glycerin | 3.00 | 3.00 | 3.00 | 3.00 | 3.00 | 3.00 | 3.00 |
| Magnesium Sulfate | 2.00 | 2.00 | 2.00 | 2.00 | 2.00 | 2.00 | 2.00 |
| Alcohol | 8.00 | 8.00 | 8.00 | 8.00 | 8.00 | 8.00 | 8.00 |
| Phenoxyethanol, | 1.00 | 1.00 | 1.00 | 1.00 | 1.00 | 1.00 | 1.00 |
| Butyl- | | | | | | | |
| paraben, Ethylparaben, | | | | | | | |
| Propylparaben, | | | | | | | |
| Methyl- | | | | | | | |
| paraben | | | | | | | |
| Aqua (water) | 38.00 | 36.40 | 37.00 | 34.40 | 38.10 | 36.60 | 37.10 |
| Complete | 100.00 | 100.00 | 100.00 | 100.00 | 100.00 | 100.00 | 100.00 |
| | INCI | | | | | | |
| | ST-16-49 % | ST-16-50 % | ST-16-51 % | ST-16-52 % | ST-16-53 % | ST-16-54 % | ST-16-55 % |
| Phase A | | | | | | | |
| Bis-PEG/PPG-14/14 | 1.50 | 1.50 | 1.50 | 1.50 | 1.50 | 1.50 | 1.50 |
| Dimethicone, | | | | | | | |
| Cyclopentasiloxane | | | | | | | |
| Cetyl PEG/PPG-10/1 | 1.30 | 1.30 | 1.30 | 1.30 | 1.30 | 1.30 | 1.30 |
| Dimethicone | | | | | | | |
| Cyclomethicone | 13.00 | 13.00 | 13.00 | 13.00 | 13.00 | 13.00 | 13.00 |
| Ethylhexyl Palmitate | 2.00 | 2.00 | 2.00 | 2.00 | 2.00 | 2.00 | 2.00 |
| Cyclopentasiloxane, | 5.00 | 5.00 | 5.00 | 5.00 | 5.00 | 5.00 | 5.00 |
| Dimethicone/Vinyl- | | | | | | | |
| dimethicone, | | | | | | | |
| Crosspolymer | | | | | | | |
| Diethylhexyl | 5.00 | 5.00 | 5.00 | 5.00 | 5.00 | 5.00 | 5.00 |
| Carbonate | | | | | | | |
| Perfume | 0.30 | 0.30 | 0.30 | 0.30 | 0.30 | 0.30 | 0.30 |
| Phase B | | | | | | | |
| Dihydroxyacetone | | 5.00 | | 5.00 | | 1.00 | 2.00 |
| Dihydroxyacetone, | 2.00 | | 5.00 | | 5.00 | | |
| Sodium Metabisulfite, | | | | | | | |
| Magnesium stearate | | | | | | | |
| Sodium Metabisulfite | | 0.50 | | 0.50 | | 0.10 | 0.20 |
| Troxerutin | 1.00 | 2.50 | 2.50 | 2.50 | 2.50 | 0.50 | 1.00 |
| Colorona ® Bronze | 2.00 | | | 5.00 | 5.00 | | |
| Erythrulose | | | | | | 2.00 | 2.00 |
| Ectoine | 0.30 | 0.30 | 0.30 | 0.30 | 0.30 | 0.30 | 0.30 |
| Propylene Glycol | 18.00 | 18.00 | 18.00 | 18.00 | 18.00 | 18.00 | 18.00 |
| Glycerin | 3.00 | 3.00 | 3.00 | 3.00 | 3.00 | 3.00 | 3.00 |
| Magnesium Sulfate | 2.00 | 2.00 | 2.00 | 2.00 | 2.00 | 2.00 | 2.00 |
| Alcohol | 8.00 | 8.00 | 8.00 | 8.00 | 8.00 | 8.00 | 8.00 |
| Phenoxyethanol, | 1.00 | 1.00 | 1.00 | 1.00 | 1.00 | 1.00 | 1.00 |
| Butyl- | | | | | | | |
| paraben, Ethylparaben, | | | | | | | |
| Propylparaben, | | | | | | | |
| Methyl- | | | | | | | |
| paraben | | | | | | | |
| Aqua (water) | 34.60 | 31.60 | 32.10 | 26.60 | 27.10 | 36.00 | 34.40 |
| Complete | 100.00 | 100.00 | 100.00 | 100.00 | 100.00 | 100.00 | 100.00 |
| | INCI | | | | | | |
| | ST-16-56 % | ST-16-57 % | ST-16-58 % | ST-16-59 % | ST-16-60 % | ST-16-61 % | ST-16-62 % |
| Phase A | | | | | | | |
| Bis-PEG/PPG-14/14 | 1.50 | 1.50 | 1.50 | 1.50 | 1.50 | 1.50 | 1.50 |
| Dimethicone, | | | | | | | |
| Cyclopentasiloxane | | | | | | | |
| Cetyl PEG/PPG-10/1 | 1.30 | 1.30 | 1.30 | 1.30 | 1.30 | 1.30 | 1.30 |
| Dimethicone | | | | | | | |
| Cyclomethicone | 13.00 | 13.00 | 13.00 | 13.00 | 13.00 | 13.00 | 13.00 |
| Ethylhexyl Palmitate | 2.00 | 2.00 | 2.00 | 2.00 | 2.00 | 2.00 | 2.00 |
| Cyclopentasiloxane, | 5.00 | 5.00 | 5.00 | 5.00 | 5.00 | 5.00 | 5.00 |
| Dimethicone/Vinyl- | | | | | | | |
| dimethicone, | | | | | | | |
| Crosspolymer | | | | | | | |
| Diethylhexyl | 5.00 | 5.00 | 5.00 | 5.00 | 5.00 | 5.00 | 5.00 |
| Carbonate | | | | | | | |
| Perfume | 0.30 | 0.30 | 0.30 | 0.30 | 0.30 | 0.30 | 0.30 |
| Phase B | | | | | | | |
| Dihydroxyacetone | | | 5.00 | 5.00 | | | 1.00 |
| Dihydroxyacetone, | 1.00 | 2.00 | | | 5.00 | 5.00 | |
| Sodium Metabisulfite, | | | | | | | |
| Magnesium stearate | | | | | | | |
| Sodium Metabisulfite | | | 0.50 | 0.50 | | | |
| Troxerutin | 0.50 | 1.00 | 2.50 | 2.50 | 2.50 | 2.50 | 0.50 |
| Colorona ® Bronze | | | | 5.00 | | 5.00 | 1.00 |
| Erythrulose | 2.00 | 2.00 | 2.00 | 2.00 | 2.00 | 2.00 | |
| Ectoine | 0.30 | 0.30 | 0.30 | 0.30 | 0.30 | 0.30 | 0.30 |
| Propylene Glycol | 18.00 | 18.00 | 18.00 | 18.00 | 18.00 | 18.00 | 18.00 |
| Glycerin | 3.00 | 3.00 | 3.00 | 3.00 | 3.00 | 3.00 | 3.00 |
| Magnesium Sulfate | 2.00 | 2.00 | 2.00 | 2.00 | 2.00 | 2.00 | 2.00 |
| Alcohol | 8.00 | 8.00 | 8.00 | 8.00 | 8.00 | 8.00 | 8.00 |
| Phenoxyethanol, | 1.00 | 1.00 | 1.00 | 1.00 | 1.00 | 1.00 | 1.00 |
| Butyl- | | | | | | | |
| paraben, Ethylparaben, | | | | | | | |
| Propylparaben, | | | | | | | |
| Methyl- | | | | | | | |
| paraben | | | | | | | |
| Aqua (water) | 36.10 | 34.60 | 29.60 | 24.60 | 30.10 | 25.10 | 37.10 |
| Complete | 100.00 | 100.00 | 100.00 | 100.00 | 100.00 | 100.00 | 100.00 |

| | INCI | | | | |
|---|---|---|---|---|---|
| | ST-16-63 % | ST-16-64 % | ST-16-65 % | ST-16-66 % | ST-16-67 % |
| Phase A | | | | | |
| Bis-PEG/PPG-14/14 | 1.50 | 1.50 | 1.50 | 1.50 | 1.50 |
| Dimethicone, | | | | | |
| Cyclopentasiloxane | | | | | |
| Cetyl PEG/PPG-10/1 | 1.30 | 1.30 | 1.30 | 1.30 | 1.30 |
| Dimethicone | | | | | |
| Cyclomethicone | 13.00 | 13.00 | 13.00 | 13.00 | 13.00 |
| Ethylhexyl Palmitate | 2.00 | 2.00 | 2.00 | 2.00 | 2.00 |
| Cyclopentasiloxane, | 5.00 | 5.00 | 5.00 | 5.00 | 5.00 |
| Dimethicone/Vinyl- | | | | | |
| dimethicone, | | | | | |
| Crosspolymer | | | | | |
| Diethylhexyl | 5.00 | 5.00 | 5.00 | 5.00 | 5.00 |
| Carbonate | | | | | |
| Perfume | 0.30 | 0.30 | 0.30 | 0.30 | 0.30 |
| Phase B | | | | | |
| Dihydroxyacetone | 2.00 | 5.00 | 1.00 | 2.00 | 5.00 |
| Dihydroxyacetone, | | | | | 5.00 |
| Sodium Metabisulfite, | | | | | |
| Magnesium stearate | | | | | |
| Sodium Metabisulfite | | | | | |
| Troxerutin | 1.00 | 2.50 | 0.50 | 1.00 | 2.50 |
| Colorona ® Bronze | 2.00 | 5.00 | 1.00 | 2.00 | 5.00 |
| Erythrulose | | | 0.50 | 1.00 | 2.50 |
| Ectoine | 0.30 | 0.30 | 0.30 | 0.30 | 0.30 |
| Propylene Glycol | 18.00 | 18.00 | 17.00 | 17.00 | 17.00 |
| Glycerin | 3.00 | 3.00 | 3.00 | 3.00 | 3.00 |
| Magnesium Sulfate | 2.00 | 2.00 | 2.00 | 2.00 | 2.00 |
| Alcohol | 8.00 | 8.00 | 8.00 | 8.00 | 8.00 |
| Phenoxyethanol, | 1.00 | 1.00 | 1.00 | 1.00 | 1.00 |
| Butyl- | | | | | |
| paraben, Ethylparaben, | | | | | |
| Propylparaben, | | | | | |
| Methyl- | | | | | |
| paraben | | | | | |
| Aqua (water) | 34.60 | 27.10 | 37.60 | 34.60 | 25.60 |
| Complete | 100.00 | 100.00 | 100.00 | 100.00 | 100.00 |

Process:

Firstly, magnesium sulfate is stirred into the water of phase B, and the further ingredients of phase B are subsequently added. Phase B is subsequently added slowly to phase A with stirring, and the mixture is homogenised.

### EXAMPLE 13

### O/W Cream with Troxerutin

| | INCI | | | | | | |
|---|---|---|---|---|---|---|---|
| | ST-08-29 % | ST-08-30 % | ST-08-31 % | ST-08-32 % | ST-08-33 % | ST-08-34 % | ST-08-35 % |
| Phase A | | | | | | | |
| Glyceryl Stearate, | 8.00 | 8.00 | 8.00 | 8.00 | 8.00 | 8.00 | 8.00 |
| Steareth-26, Ceteth-20, | | | | | | | |
| Stearyl Alcohol | | | | | | | |
| Cetearyl Alcohol | 1.50 | 1.50 | 1.50 | 1.50 | 1.50 | 1.50 | 1.50 |
| Cetearyl Ethylhexanoate | 5.50 | 5.50 | 5.50 | 5.50 | 5.50 | 5.50 | 5.50 |
| Caprylic/Capric | 5.50 | 5.50 | 5.50 | 5.50 | 5.50 | 5.50 | 5.50 |
| Triglyceride | | | | | | | |
| Stearoxy Dimethicone | 1.00 | 1.00 | 1.00 | 1.00 | 1.00 | 1.00 | 1.00 |
| Dimethicone | 0.50 | 0.50 | 0.50 | 0.50 | 0.50 | 0.50 | 0.50 |
| Tocopheryl Acetate | 0.50 | 0.50 | 0.50 | 0.50 | 0.50 | 0.50 | 0.50 |
| Propylparaben | 0.05 | 0.05 | 0.05 | 0.05 | 0.05 | 0.05 | 0.05 |
| Phase B | | | | | | | |
| Ectoine | 0.30 | 0.30 | 0.30 | 0.30 | 0.30 | 0.30 | 0.30 |
| Propylene Glycol | 3.00 | 3.00 | 3.00 | 3.00 | 3.00 | 3.00 | 3.00 |
| Methylparaben | 0.15 | 0.15 | 0.15 | 0.15 | 0.15 | 0.15 | 0.15 |
| Aqua (water) | 62.30 | 60.70 | 61.30 | 58.70 | 62.40 | 60.90 | 61.40 |
| Phase C | | | | | | | |
| Dihydroxyacetone | 1.00 | 2.00 | 1.00 | 2.00 | | | |
| Dihydroxyacetone, | | | | | 1.00 | 2.00 | 1.00 |
| Sodium Metabisulfite, | | | | | | | |
| Magnesium Stearate | | | | | | | |
| Sodium Metabisulfite | 0.10 | 0.20 | 0.10 | 0.20 | | | |
| Troxerutin | 0.50 | 1.00 | 0.50 | 1.00 | 0.50 | 1.00 | 0.50 |
| Colorona ® Bronze | | | 1.00 | 2.00 | | | 1.00 |
| Erythrulose | | | | | | | |
| Aqua (water) | 10.00 | 10.00 | 10.00 | 10.00 | 10.00 | 10.00 | 10.00 |
| Phase D | | | | | | | |
| Perfume | 0.10 | 0.10 | 0.10 | 0.10 | 0.10 | 0.10 | 0.10 |
| Complete | 100.00 | 100.00 | 100.00 | 100.00 | 100.00 | 100.00 | 100.00 |
| | INCI | | | | | | |
| | ST-08-36 % | ST-08-37 % | ST-08-38 % | ST-08-39 % | ST-08-40 % | ST-08-41 % | ST-08-42 % |
| Phase A | | | | | | | |
| Glyceryl Stearate, | 8.00 | 8.00 | 8.00 | 8.00 | 8.00 | 8.00 | 8.00 |
| Steareth-26, Ceteth-20, | | | | | | | |
| Stearyl Alcohol | | | | | | | |
| Cetearyl Alcohol | 1.50 | 1.50 | 1.50 | 1.50 | 1.50 | 1.50 | 1.50 |
| Cetearyl Ethylhexanoate | 5.50 | 5.50 | 5.50 | 5.50 | 5.50 | 5.50 | 5.50 |
| Caprylic/Capric | 5.50 | 5.50 | 5.50 | 5.50 | 5.50 | 5.50 | 5.50 |
| Triglyceride | | | | | | | |
| Stearoxy Dimethicone | 1.00 | 1.00 | 1.00 | 1.00 | 1.00 | 1.00 | 1.00 |
| Dimethicone | 0.50 | 0.50 | 0.50 | 0.50 | 0.50 | 0.50 | 0.50 |
| Tocopheryl Acetate | 0.50 | 0.50 | 0.50 | 0.50 | 0.50 | 0.50 | 0.50 |
| Propylparaben | 0.05 | 0.05 | 0.05 | 0.05 | 0.05 | 0.05 | 0.05 |
| Phase B | | | | | | | |
| Ectoine | 0.30 | 0.30 | 0.30 | 0.30 | 0.30 | 0.30 | 0.30 |
| Propylene Glycol | 3.00 | 3.00 | 3.00 | 3.00 | 3.00 | 3.00 | 3.00 |
| Methylparaben | 0.15 | 0.15 | 0.15 | 0.15 | 0.15 | 0.15 | 0.15 |
| Aqua (water) | 58.90 | 55.90 | 56.40 | 50.90 | 51.40 | 60.30 | 58.70 |
| Phase C | | | | | | | |
| Dihydroxyacetone | | 5.00 | | 5.00 | | 1.00 | 2.00 |
| Dihydroxyacetone, | 2.00 | | 5.00 | | 5.00 | | |
| Sodium Metabisulfite, | | | | | | | |
| Magnesium Stearate | | | | | | | |
| Sodium Metabisulfite | | 0.50 | | 0.50 | | 0.10 | 0.20 |
| Troxerutin | 1.00 | 2.50 | 2.50 | 2.50 | 2.50 | 0.50 | 1.00 |
| Colorona ® Bronze | 2.00 | | | 5.00 | 5.00 | | |
| Erythrulose | | | | | | 2.00 | 2.00 |
| Aqua (water) | 10.00 | 10.00 | 10.00 | 10.00 | 10.00 | 10.00 | 10.00 |
| Phase D | | | | | | | |
| Perfume | 0.10 | 0.10 | 0.10 | 0.10 | 0.10 | 0.10 | 0.10 |
| Complete | 100.00 | 100.00 | 100.00 | 100.00 | 100.00 | 100.00 | 100.00 |
| | INCI | | | | | | |
| | ST-08-43 % | ST-08-44 % | ST-08-45 % | ST-08-46 % | ST-08-47 % | ST-08-48 % | ST-08-49 % |
| Phase A | | | | | | | |
| Glyceryl Stearate, | 8.00 | 8.00 | 8.00 | 8.00 | 8.00 | 8.00 | 8.00 |
| Steareth-26, Ceteth-20, | | | | | | | |
| Stearyl Alcohol | | | | | | | |
| Cetearyl Alcohol | 1.50 | 1.50 | 1.50 | 1.50 | 1.50 | 1.50 | 1.50 |
| Cetearyl Ethylhexanoate | 5.50 | 5.50 | 5.50 | 5.50 | 5.50 | 5.50 | 5.50 |
| Caprylic/Capric | 5.50 | 5.50 | 5.50 | 5.50 | 5.50 | 5.50 | 5.50 |
| Triglyceride | | | | | | | |
| Stearoxy Dimethicone | 1.00 | 1.00 | 1.00 | 1.00 | 1.00 | 1.00 | 1.00 |
| Dimethicone | 0.50 | 0.50 | 0.50 | 0.50 | 0.50 | 0.50 | 0.50 |
| Tocopheryl Acetate | 0.50 | 0.50 | 0.50 | 0.50 | 0.50 | 0.50 | 0.50 |
| Propylparaben | 0.05 | 0.05 | 0.05 | 0.05 | 0.05 | 0.05 | 0.05 |
| Phase B | | | | | | | |
| Ectoine | 0.30 | 0.30 | 0.30 | 0.30 | 0.30 | 0.30 | 0.30 |
| Propylene Glycol | 3.00 | 3.00 | 3.00 | 3.00 | 3.00 | 3.00 | 3.00 |
| Methylparaben | 0.15 | 0.15 | 0.15 | 0.15 | 0.15 | 0.15 | 0.15 |
| Aqua (water) | 60.40 | 58.90 | 53.90 | 48.990 | 54.40 | 49.40 | 61.40 |
| Phase C | | | | | | | |
| Dihydroxyacetone | | | 5.00 | 5.00 | | | 1.00 |
| Dihydroxyacetone, | 1.00 | 2.00 | | | 5.00 | 5.00 | |
| Sodium Metabisulfite, | | | | | | | |
| Magnesium Stearate | | | | | | | |
| Sodium Metabisulfite | | | 0.50 | 0.50 | | | |
| Troxerutin | 0.50 | 1.00 | 2.50 | 2.50 | 2.50 | 2.50 | 0.50 |
| Colorona ® Bronze | | | | 5.00 | | 5.00 | 1.00 |
| Erythrulose | 2.00 | 2.00 | 2.00 | 2.00 | 2.00 | 2.00 | |
| Aqua (water) | 10.00 | 10.00 | 10.00 | 10.00 | 10.00 | 10.00 | 10.00 |
| Phase D | | | | | | | |
| Perfume | 0.10 | 0.10 | 0.10 | 0.10 | 0.10 | 0.10 | 0.10 |
| Complete | 100.00 | 100.00 | 100.00 | 100.00 | 100.00 | 100.00 | 100.00 |

| | INCI | | | | |
|---|---|---|---|---|---|
| | ST-08-50 % | ST-08-51 % | ST-08-52 % | ST-08-53 % | ST-08-54 % |
| Phase A | | | | | |
| Glyceryl Stearate, | 8.00 | 8.00 | 8.00 | 8.00 | 8.00 |
| Steareth-26, Ceteth-20, | | | | | |
| Stearyl Alcohol | | | | | |
| Cetearyl Alcohol | 1.50 | 1.50 | 1.50 | 1.50 | 1.50 |
| Cetearyl Ethylhexanoate | 5.50 | 5.50 | 5.50 | 5.50 | 5.50 |
| Caprylic/Capric | 5.50 | 5.50 | 5.50 | 5.50 | 5.50 |
| Triglyceride | | | | | |
| Stearoxy Dimethicone | 1.00 | 1.00 | 1.00 | 1.00 | 1.00 |
| Dimethicone | 0.50 | 0.50 | 0.50 | 0.50 | 0.50 |
| Tocopheryl Acetate | 0.50 | 0.50 | 0.50 | 0.50 | 0.50 |
| Propylparaben | 0.05 | 0.05 | 0.05 | 0.05 | 0.05 |
| Phase B | | | | | |
| Ectoine | 0.30 | 0.30 | 0.30 | 0.30 | 0.30 |
| Propylene Glycol | 3.00 | 3.00 | 3.00 | 3.00 | 3.00 |
| Methylparaben | 0.15 | 0.15 | 0.15 | 0.15 | 0.15 |
| Aqua (water) | 58.90 | 51.40 | 60.90 | 57.90 | 48.90 |
| Phase C | | | | | |
| Dihydroxyacetone | 2.00 | 5.00 | 1.00 | 2.00 | 5.00 |
| Dihydroxyacetone, | | | | | |
| Sodium Metabisulfite, | | | | | |
| Magnesium Stearate | | | | | |
| Sodium Metabisulfite | | | | | |
| Troxerutin | 1.00 | 2.50 | 0.50 | 1.00 | 2.50 |
| Colorona ® Bronze | 2.00 | 5.00 | 1.00 | 2.00 | 5.00 |
| Erythrulose | | | 0.50 | 1.00 | 2.50 |
| Aqua (water) | 10.00 | 10.00 | 10.00 | 10.00 | 10.00 |
| Phase D | | | | | |
| Perfume | 0.10 | 0.10 | 0.10 | 0.10 | 0.10 |
| Complete | 100.00 | 100.00 | 100.00 | 100.00 | 100.00 |

Process:

Phases A and B are heated separately to 80° C. Phase B is then added slowly to phase A with stirring and homogenised. The mixture is cooled with stirring, and phase C is added at 40° C., and phase D is then added.

1. A method of improving and/or stabilizing the odor of a composition comprising adding at least one flavonoid to said composition.

2. Method according to claim 1, characterised in that the odour improvement takes place in cosmetic, dermatological or pharmaceutical compositions, premixes for cosmetic, dermatological or pharmaceutical compositions or foods or food supplements.

3. Method according to claim 1, characterised in that the flavonoid is selected from the group of the flavonols, flavonol o-glycosides, flavonolor flavonol o-glycoside-containing extracts.

4. Method according to claim 1, characterised in that the at least one flavonoid is quercetin, rutin, rutin sulfate, α-glucosylrutin, tiliroside, troxerutin and/or isoquercetin.

5. Method according to claim 1, characterised in that the flavonoid is a flavonoid in which one or more phenolic hydroxyl groups have been blocked by etherification or esterification.

6. Method according to claim 5, characterised in that the flavonoid is a hydroxyethyl-substituted flavonoid, preferably troxerutin, troxequercetin, troxeisoquercetin or troxeluteolin.

7. Method according to claim 5, characterised in that the flavonoid is a flavonoid sulfate or flavonoid phosphate, preferably a rutin sulfate.

8. Method according to claim 1, characterised in that the odour improvement takes place in a composition comprising aldehydes or ketones, such as, in particular, dihydroxyacetone or vanillin.

9. Method according to claim 1, characterised in that the at least one flavonoid is employed in a total amount of 0.01 to 10% by weight, based on the odour-stabilised composition.

10. Method according to claim 1, characterised in that the at least one flavonoid is employed in a total amount of 1 to 95% by weight, based on the odour-stabilised premix for a composition.

11. Method according to claim 1, characterised in that the at least one flavonoid stabilises and/or improves the odour of compositions comprising at least one self-tanning substance on the skin.

12. Method according to claim 11, characterised in that the at least one self-tanning substance is selected from dihydroxyacetone and/or erythrulose.

13. Perfume composition, characterised in that the composition comprises at least one flavonoid in addition to fragrances.

14. Composition according to claim 13, characterised in that the at least one flavonoid is present in the composition in a total amount of 0.01 to 10% by weight.

15. Composition according to claim 13, characterised in that the flavonoid is a flavonoid in which one or more phenolic hydroxyl groups have been blocked by etherification or esterification.

16. Process for the preparation of a perfume composition, characterised in that at least one flavonoid is mixed with fragrances.
